(19) United States
(12) Patent Application Publication
(10) Pub. No.: US 2010/0063301 A1
(43) Pub. Date: Mar. 11, 2010

(54) SYNTHESIS OF THROMBOPOIETIN ACTIVITY MODULATING COMPOUNDS

(75) Inventor: Dean Phillips, San Marcos, CA (US)

Correspondence Address: KNOBBE MARTENS OLSON & BEAR LLP, 2040 MAIN STREET, FOURTEENTH FLOOR, IRVINE, CA, 92614, US

(73) Assignee: LIGAND PHARMACEUTICALS INC, SAN DIEGO, CA (US)

(21) Appl. No.: 12/282,876

(22) PCT Filed: Mar. 14, 2007
(86) PCT No.: PCT/US07/06547

§ 371 Date: Jan. 21, 2009

Related U.S. Application Data
(60) Provisional application No. 60/782,426, filed on Mar. 15, 2006.

(51) Int. Cl. C07D 209/04

(52) U.S. Cl. 548/483

(57) ABSTRACT

Disclosed herein are various methods for synthesizing compounds that modulate thrombopoietin activity. Also disclosed are intermediates useful for the preparation of these compounds.

### FIELD OF THE INVENTION

This invention relates to compounds and methods for making compounds that modulate one or more thrombopoietin activity and/or bind to thrombopoietin receptors.

### BACKGROUND

Thrombopoietin (TPO), also referred to as c-Mpl ligand, mpl ligand, megapoietin, and megakaryocyte growth and development factor, is a glycoprotein that has been shown to be involved in production of platelets. See e.g., Wendling, F., et. al., Biotherapy 10(4):269-77 (1998); Kuter D. J. et al., The Oncologist, 1:98-106 (1996); and Metcalf, Nature 369: 519-520 (1994). TPO has been cloned and its amino acid sequence and the cDNA sequence encoding it have been described. See e.g., U.S. Pat. No. 5,766,581; Kuter, D. J. et al., Proc. Natl. Acad. Sci., 91:11104-11108 (1994); de Sauvage F. V., et al., Nature, 369: 533-538 (1994); Lok, S. et al., Nature 369:565-568 (1994); and Wending, F. et al., Nature, 369: 571-574 (1994).

In certain instances, TPO activity results from binding of TPO to the TPO receptor (also called MPL). The TPO receptor has been cloned and its amino acid sequence has been described. See e.g., Vigon et al., Proc. Natl. Acad. Sci., 89:5640-5644 (1992).

In certain instances, TPO modulators may be useful in treating a variety of hematopoietic conditions, including, but not limited to, thrombocytopenia. See e.g., Baser et al. Blood 89:3118-3128 (1997); Fanucchi et al. New Engl. J. Med. 336:404-409 (1997). For example, patients undergoing certain chemotherapies, including but not limited to chemotherapy and/or radiation therapy for the treatment of cancer, may have reduced platelet levels. In certain instances, treating such patients with a selective TPO modulator increases platelet levels. In certain instances, selective TPO modulators stimulate production of glial cells, which may result in repair of damaged nerve cells.

Certain TPO mimics have been described previously. See e.g., U.S. application Ser. No. 11/256,572, filed on Oct. 21, 2005 and entitled “THROMBOPOIETIN ACTIVITY MODULATING COMPOUNDS AND METHODS;” WO 03/103686A1, filed Jun. 6, 2003 and entitled “THROMBOPOIETIN MIMETICS;” and WO 01/21180, filed Sep. 22, 2000 and entitled “THROMBOPOIETIN MIMETICS,” each of which is hereby incorporated in its entirety for any reason.

### SUMMARY OF THE INVENTION

In certain embodiments, the present invention provides methods for making compounds of Formula I, II, and/or III:

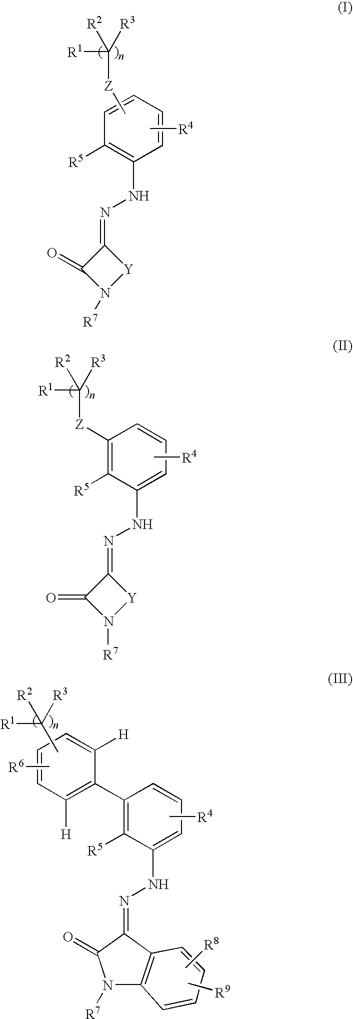

or a pharmaceutically acceptable salt, ester, amide, or prodrug thereof, wherein R¹-R⁹ are as described herein. In certain embodiments, the present invention provides intermediate compounds useful for making compounds of Formula I, II, and/or III.

In certain embodiments, the present invention provides a compound having the structure:

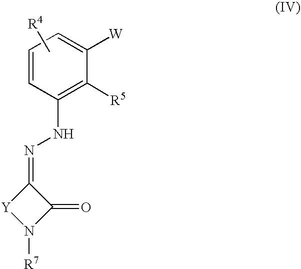

wherein:
R⁴ is selected from hydrogen, F, Cl, Br, C₁-C₄ aliphatic, C₁-C₄ haloaliphatic, C₁-C₄ heteroaliphatic, and a ring;
R⁵ is selected from hydrogen, OR¹⁰, SR¹⁰, NHR¹¹, and CO₂H;
R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R¹⁰ is selected from hydrogen, a protecting group, an optionally substituted C₁-C₄ aliphatic, an optionally substituted C₁-C₄ haloaliphatic, an optionally substituted C₁-C₄ heteroaliphatic, and an optionally substituted ring;
R¹¹ is selected from hydrogen, SO₂R¹⁵, C₁-C₄ aliphatic, C₁-C₄ haloaliphatic, C₁-C₄ heteroaliphatic, and a ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
Y is a 1-4 atom spacer comprising one or more groups selected from an optionally substituted C₁-C₆ aliphatic, an optionally substituted C₁-C₆ heteroaliphatic, an optionally substituted phenyl, an optionally substituted heteroaryl, an optionally substituted C₃-C₅ heterocycle, and an optionally substituted alicyclic;
W is selected from a halogen, B(OH)₂, B(OR^{A})₂, Sn(R^{B})₃ where each R^{A} is selected from an optionally substituted C₁-C₆ aliphatic; or the two OR^{A} groups together form an optionally substituted ring; and R^{B} is selected from an optionally substituted C₁-C₆ aliphatic, or an optionally substituted phenyl, or an optionally substituted heteroaryl;
m is 0, 1, or 2.

In certain embodiments, the present invention provides a compound of Formula IV, wherein:
Y is selected from:

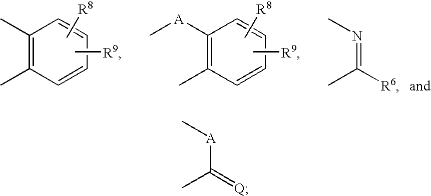

Q is selected from O and S;
A is selected from O, S, NR¹⁰, and CR¹⁰R¹⁰; and
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, C₁-C₄ heterohaloaliphatic, and a ring, wherein R¹⁰ and R¹⁴ are as described above.

In certain embodiments, the present invention provides a compound having the structure:

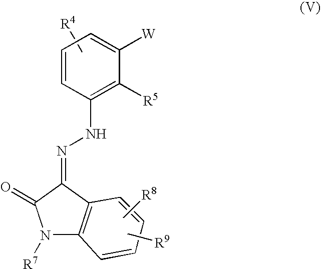

wherein:
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, heterohaloaliphatic, and a ring, wherein R¹⁰ and R¹⁴ are as described above.

In certain embodiments, the present invention provides a compound having the structure:

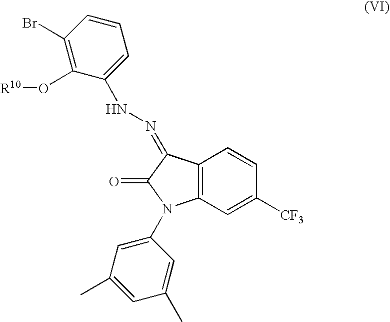

wherein R¹⁰ is hydrogen, a protecting group, an optionally substituted C₁-C₄ aliphatic, an optionally substituted C₁-C₄ haloaliphatic, or an optionally substituted C₁-C₄ heteroaliphatic.

In certain embodiments, the present invention provides a method of obtaining a compound having the structure:

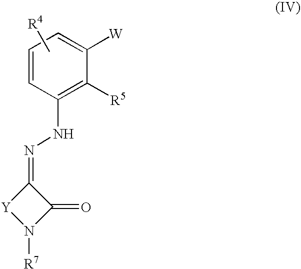

comprising reacting a compound having the structure:

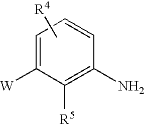

with a nitrite and a compound having the structure:

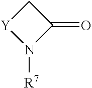

wherein:
R⁴ is selected from hydrogen, F, Cl, Br, C₁-C₄ aliphatic, C₁-C₄ haloaliphatic, heteroaliphatic, and a ring;
R⁵ is selected from hydrogen, OR¹⁰, SR¹⁰, NHR¹¹, and CO₂H;
R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R¹⁰ is selected from hydrogen, a protecting group, an optionally substituted C₁-C₄ aliphatic, an optionally substituted C₁-C₄ haloaliphatic, an optionally substituted C₁-C₄ heteroaliphatic, and an optionally substituted ring;
R¹¹ is selected from hydrogen, SO₂R¹⁵, C₁-C₄ aliphatic, C₁-C₄ haloaliphatic, C₁-C₄ heteroaliphatic, and a ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
Y is a 1-4 atom spacer comprising one or more groups selected from an optionally substituted C₁-C₆ aliphatic, an optionally substituted C₁-C₆ heteroaliphatic, an optionally substituted phenyl, an optionally substituted heteroaryl, an optionally substituted C₃-C₅ heterocycle, and an optionally substituted alicyclic;
W is selected from a halogen, B(OH)₂, B(OR^{A})₂, Sn(R^{B})₃ where each R^{A} is selected from an optionally substituted C₁-C₆ aliphatic; or the two OR^{A} groups together form an optionally substituted ring; and R^{B} is selected from an optionally substituted C₁-C₆ aliphatic, or an optionally substituted phenyl, or an optionally substituted heteroaryl; and
m is 0, 1, or 2.

In certain such embodiments, Y is selected from:

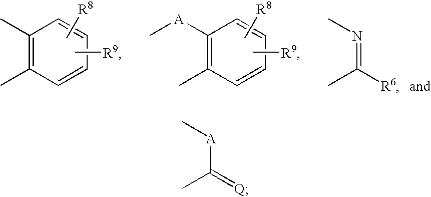

Q is selected from O and S;
A is selected from O, S, NR¹⁰, and CR¹⁰R¹⁰; and
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, C₁-C₄ heterohaloaliphatic, and a ring, wherein R¹⁰ and R¹⁴ are as described above.

In certain embodiments, the present invention provides a method of obtaining a compound having the structure:

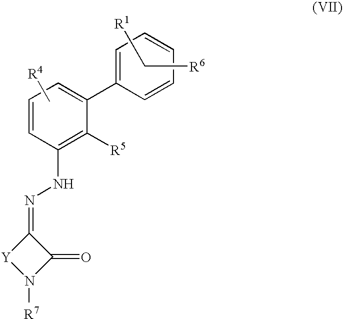

comprising reacting a compound having the structure:

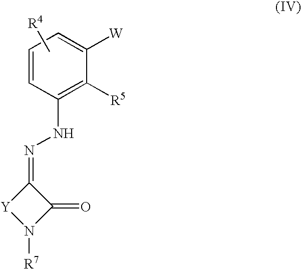

with a compound having the structure:

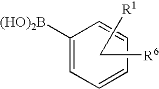

wherein:
R¹ is selected from CO₂R¹⁰, CONR¹⁰R¹¹, SO₃R¹⁰, and a carboxylic acid bioisostere;
R⁴ is selected from hydrogen, F, Cl, Br, C₁-C₄ aliphatic, C₁-C₄ haloaliphatic, C₁-C₄ heteroaliphatic, and a ring;
R⁵ is selected from hydrogen, OR¹⁰, SR¹⁰, NHR¹¹, and CO₂H;
R⁶ is selected from hydrogen, OR¹², NR¹²R¹³, F, Cl, Br, C₁-C₄ alkyl, C₁-C₄ haloalkyl, C₁-C₄ heteroalkyl, and a ring;
R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R¹⁰ is selected from hydrogen, a protecting group, an optionally substituted C₁-C₄ aliphatic, an optionally substituted C₁-C₄ haloaliphatic, an optionally substituted C₁-C₄ heteroaliphatic, and an optionally substituted ring;
R¹¹ is selected from hydrogen, SO₂R¹⁵, C₁-C₄ aliphatic, C₁-C₄ haloaliphatic, C₁-C₄ heteroaliphatic, and a ring;
R¹² and R¹³ are each independently selected from hydrogen, an optionally substituted C₁-C₄ aliphatic, an optionally substituted C₁-C₄ haloaliphatic, an optionally substituted C₁-C₄ heteroaliphatic, an optionally substituted ring, and (CH₂)ₘR¹⁴; or one of R¹² and R¹³ is an optionally substituted C₂-C₆ aliphatic or an optionally substituted ring and the other of R¹² and R¹³ is null; or R¹² and R¹³ are linked to form an optionally substituted C₃-C₈ ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
Y is a 1-4 atom spacer comprising one or more groups selected from an optionally substituted C₁-C₆ aliphatic, an optionally substituted C₁-C₆ heteroaliphatic, an optionally substituted phenyl, an optionally substituted heteroaryl, an optionally substituted C₃-C₅ heterocycle, and an optionally substituted alicyclic;
W is selected from a halogen, B(OH)₂, B(OR^{A})₂, Sn(R^{B})₃ where each R^{A} is selected from an optionally substituted C₁-C₆ aliphatic; or the two OR^{A} groups together form an optionally substituted ring; and R⁸ is selected from an optionally substituted C₁-C₆ aliphatic, or an optionally substituted phenyl, or an optionally substituted heteroaryl; and
m is 0, 1, or 2.

In certain embodiments, the present invention provides a method of making a compound that is made is a selective TPO modulator; a selective TPO receptor agonist; a selective TPO receptor antagonist; a selective TPO partial agonist; a selective TPO receptor binding compound; a TPO mimic; and/or a tissue-selective selective TPO modulator.

In certain embodiments, the present invention provides a compound useful for making a selective TPO modulator; a selective TPO receptor agonist; a selective TPO receptor antagonist; a selective TPO partial agonist; a selective TPO receptor binding compound; a TPO mimic; and/or a tissue-selective selective TPO modulator.

### DETAILED DESCRIPTION OF THE PREFERRED EMBODIMENTS

It is to be understood that both the foregoing general description and the following detailed description are exemplary and explanatory only and are not restrictive of the invention claimed. Herein, the use of the singular includes the plural unless specifically stated otherwise. Herein, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### DEFINITIONS

Unless specific definitions are provided, the nomenclatures utilized in connection with, and the laboratory procedures and techniques of, analytical chemistry, synthetic organic chemistry, and medicinal and pharmaceutical chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, pharmaceutical preparation, formulation, and delivery, and treatment of patients. Standard techniques may be used for recombinant DNA, oligonucleotide synthesis, and tissue culture and transformation (e.g., electroporation, lipofection). Reactions and purification techniques may be performed e.g., using kits according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein for any purpose.

As used herein, the following terms are defined with the following meanings, unless expressly stated otherwise.

The term “selective binding compound” refers to a compound that selectively binds to any portion of one or more target.

The term “selective TPO receptor binding compound” refers to a compound that selectively binds to any portion of a TPO receptor.

The term “selectively binds” refers to the ability of a selective binding compound to bind to a target receptor with greater affinity than it binds to a non-target receptor. In certain embodiments, specific binding refers to binding to a target with an affinity that is at least 10, 50, 100, 250, 500, or 1000 times greater than the affinity for a non-target.

The term “target receptor” refers to a receptor or a portion of a receptor capable of being bound by a selective binding compound. In certain embodiments, a target receptor is a TPO receptor.

The term “modulator” refers to a compound that alters or elicits an activity. For example, the presence of a modulator may result in an increase or decrease in the magnitude of a certain activity compared to the magnitude of the activity in the absence of the modulator. In certain embodiments, a modulator is an inhibitor, which decreases the magnitude of one or more activities. In certain embodiments, an inhibitor completely prevents one or more biological activities. In certain embodiments, a modulator is an activator, which increases the magnitude of at least one activity. In certain embodiments the presence of a modulator results in a activity that does not occur in the absence of the modulator.

The term “selective modulator” refers to a compound that selectively modulates a target activity.

The term “selective TPO modulator” refers to a compound that selectively modulates at least one TPO activity. The term selective TPO modulator includes, but is not limited to “TPO mimic” which refers to a compound, the presence of which results in at least one TPO activity.

The term “selectively modulates” refers to the ability of a selective modulator to modulate a target activity to a greater extent than it modulates a non-target activity.

The term “target activity” refers to a biological activity capable of being modulated by a selective modulator. Certain exemplary target activities include, but are not limited to, binding affinity; signal transduction; enzymatic activity; transcription of one or more genes; the proliferation and/or differentiation of cells, including, but not limited to progenitor cells; generation of platelets; and alleviation of symptoms of a disease or condition.

The term “TPO activity” refers to a biological activity that results, either directly or indirectly from the presence of TPO. Exemplary TPO activities include, but are not limited to, proliferation and or differentiation of progenitor cells to produce platelets; hematopoiesis; growth and/or development of glial cells; repair of nerve cells; and alleviation of thrombocytopenia.

The term “thrombocytopenia” refers to a condition wherein the concentration of platelets in the blood of a patient is below what is considered normal for a healthy patient. In certain embodiments, thrombocytopenia is a platelet count less than 450,000, 400,000, 350,000, 300,000, 250,000, 200,000, 150,000, 140,000, 130,000, 120,000, 110,000, 100,000, 75,000, or 50,000 platelets per microliter of blood.

The term “receptor mediated activity” refers any biological activity that results, either directly or indirectly, from binding of a ligand to a receptor.

The term “agonist” refers to a compound, the presence of which results in a biological activity of a receptor that is the same as the biological activity resulting from the presence of a naturally occurring ligand for the receptor.

The term “partial agonist” refers to a compound, the presence of which results in a biological activity of a receptor that is of the same type as that resulting from the presence of a naturally occurring ligand for the receptor, but of a lower magnitude.

The term “antagonist” refers to a compound, the presence of which results in a decrease in the magnitude of a biological activity of a receptor. In certain embodiments, the presence of an antagonist results in complete inhibition of a biological activity of a receptor.

The term “aliphatic,” alone or in combination, refers to a straight or branched chain comprising at least one carbon atom. Aliphatics include alkyls, alkenyls, and alkynyls. In certain embodiments, aliphatics are optionally substituted. Aliphatics include, but are not limited to, methyl, ethyl, propyl, isopropyl, butyl, isobutyl, tertiary butyl, pentyl, hexyl, ethenyl, propenyl, butenyl, ethynyl, butynyl, propynyl, and the like, each of which may be optionally substituted. As used herein, aliphatic is not intended to include cyclic groups.

The term “alkyl,” alone or in combination, refers to a fully saturated aliphatic. In certain embodiments, alkyls are optionally substituted. In certain embodiments, an alkyl comprises 1 to 20 carbon atoms (whenever it appears herein, a numerical range, such as “1 to 20” or “C₁-C₂₀”, refers to each integer in the given range; e.g., “C₁-C₂₀ alkyl” means that an alkyl group comprising only 1 carbon atom, 2 carbon atoms, 3 carbon atoms, etc., up to and including 20 carbon atoms). Examples of alkyls include, but are not limited to, methyl, ethyl, n-propyl, isopropyl, n-butyl, isobutyl, sec-butyl, tert-butyl, tert-amyl, pentyl, hexyl, heptyl, octyl and the like.

The term “alkenyl,” alone or in combination, refers to an aliphatic having one or more carbon-carbon double-bonds. In certain embodiments, alkenyls are optionally substituted. Examples of alkenyls include, but are not limited to, ethenyl, propenyl, 1,4-butadienyl, and the like.

The term “alkynyl,” alone or in combination, refers to an aliphatic having one or more carbon-carbon triple-bonds. In certain embodiments, alkynyls are optionally substituted. Examples of alkynyls include, but are not limited to, ethynyl, propynyl, butynyl, and the like.

The term “haloaliphatic,” alone or in combination, refers to an aliphatic in which at least one hydrogen atom is replaced with a halogen atom. In certain embodiments in which two or more hydrogen atom are replaced with halogen atoms, the halogen atoms are all the same as one another. In certain such embodiments, the halogen atoms are not all the same as one another. Haloaliphatics include haloalkyls, haloalkenyls, and haloalkynyls. In certain embodiments, haloaliphatics are optionally substituted, in addition to the hydrogen/halogen substitution. The term “haloaliphatic” also includes perhaloaliphatic, in which all of the hydrogen atoms of the aliphatic are replaced by halogen atoms. Examples of perhaloaliphatic include trichloromethyl, pentacholorethyl, etc.

The term “heteroaliphatic,” alone or in combination, refers to a group comprising an aliphatic and one or more heteroatoms. Certain heteroaliphatics are acylaliphatics, in which the one or more heteroatoms is not within an aliphatic chain. Heteroaliphatics include heteroalkyls, including, but not limited to acylalkyls; heteroalkenyls, including, but not limited to, acylalkenyls; and heteroalkynyls, including, but not limited acylalkynyls. Examples of heteraliphatics include, but are not limited to, CH₃C(═O)CH₂—, CH₃C(═O)CH₂CH₂—, CH₃CH₂C(═O)CH₂CH₂—, CH₃C(═O)CH₂CH₂CH₂—, CH₃₀CH₂CH₂—, CH₃NHCH₂—, and the like. In certain embodiments, heteroaliphatics are optionally substituted.

The term “heterohaloaliphatic” refers to a heteroaliphatic in which at least one hydrogen atom is replaced with a halogen atom. Heterohaloaliphatics include heterohaloalkyls, heterohaloalkenyls, and heterohaloalkynyls. In certain embodiments, heterohaloaliphatics are optionally substituted.

The term “olefin” refers to a C═C bond. The term “together form an olefin” refers to instances where two groups are bound to the same carbon atom and one of those two groups is ═C and the other of those two groups is null. For example, if R′ and R″ in the structure below together form an olefin:

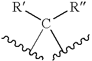

the resulting structure is:

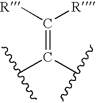

wherein R′″ and R″″ represent hydrogen. Olefins may be optional substituted, in which case R′″ and R″″ above are independently selected from hydrogen and an optional substituent.

The term “carbocycle” refers to a group comprising a covalently closed ring, wherein each of the atoms forming the ring is a carbon atom. Carbocylic rings may be formed by three, four, five, six, seven, eight, nine, or more than nine carbon atoms. Carbocycles may be optionally substituted.

The term “heterocycle” refers to a group comprising a covalently closed ring wherein at least one atom forming the ring is a carbon atom and at least one atom forming the ring is a heteroatom. Heterocyclic rings may be formed by three, four, five, six, seven, eight, nine, or more than nine atoms. Any number of those atoms may be heteroatoms (i.e., a heterocyclic ring may comprise one, two, three, four, five, six, seven, eight, nine, or more than nine heteroatoms). Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring will have additional heteroatoms in the ring. In heterocycles comprising two or more heteroatoms, those two or more heteroatoms may be the same or different from one another. Heterocycles may be optionally substituted. Binding to a heterocycle can be at a heteroatom or via a carbon atom. Examples of heterocycles include, but are not limited to the following:

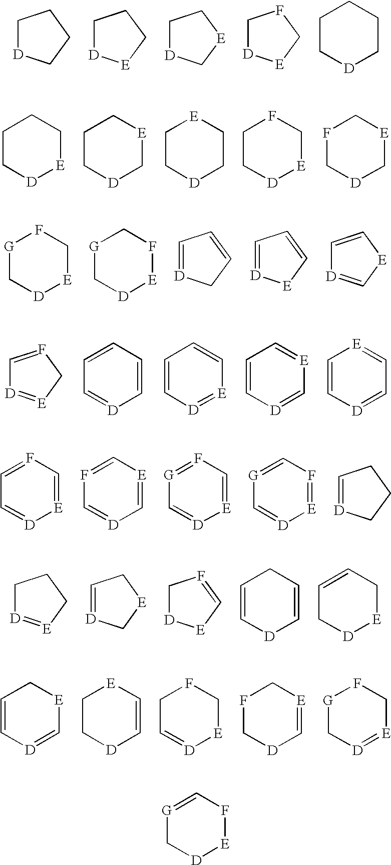

wherein D, E, F, and G independently represent a heteroatom. Each of D, E, F, and G may be the same or different from one another.

The term “heteroatom” refers to an atom other than carbon or hydrogen. Heteroatoms are typically independently selected from oxygen, sulfur, nitrogen, and phosphorus, but are not limited to those atoms. In embodiments in which two or more heteroatoms are present, the two or more heteroatoms may all be the same as one another, or some or all of the two or more heteroatoms may each be different from the others.

The term “aromatic” refers to a group comprising a covalently closed planar ring having a delocalized n-electron system comprising 4n+2π electrons, where n is an integer. Aromatic rings may be formed by five, six, seven, eight, nine, or more than nine atoms. Aromatics may be optionally substituted. Examples of aromatic groups include, but are not limited to phenyl, naphthalenyl, phenanthrenyl, anthracenyl, tetralinyl, fluorenyl, indenyl, and indanyl. The term aromatic includes, for example, benzenoid groups, connected via one of the ring-forming carbon atoms, and optionally carrying one or more substituents selected from an aryl, a heteroaryl, a cycloalkyl, a non-aromatic heterocycle, a halo, a hydroxy, an amino, a cyano, a nitro, an alkylamido, an acyl, a C₁₋₆ alkoxy, a C₁₋₆ alkyl, a C₁₋₆ hydroxyalkyl, a C₁₋₆ aminoalkyl, a C₁₋₆ alkylamino, an alkylsulfenyl, an alkylsulfinyl, an alkylsulfonyl, an sulfamoyl, or a trifluoromethyl. In certain embodiments, an aromatic group is substituted at one or more of the para, meta, and/or ortho positions. Examples of aromatic groups comprising substitutions include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyanophenyl, 4-cyanophenyl, dimethylphenyl, naphthyl, hydroxynaphthyl, hydroxymethylphenyl, (trifluoromethyl)phenyl, alkoxyphenyl, 4-morpholin-4-ylphenyl, 4-pyrrolidin-1-ylphenyl, 4-pyrazolyiphenyl, 4-triazolylphenyl, and 4-(2-oxopyrrolidin-1-yl)phenyl.

The term “aryl” refers to an aromatic ring wherein each of the atoms forming the ring is a carbon atom. Aryl rings may be formed by five, six, seven, eight, nine, or more than nine carbon atoms. Aryl groups may be optionally substituted.

The term “heteroaryl” refers to an aromatic heterocycle. Heteroaryl rings may be formed by three, four, five, six, seven, eight, nine, or more than nine atoms. Heteroaryls may be optionally substituted. Examples of heteroaryl groups include, but are not limited to, aromatic C₃₋₈ heterocyclic groups comprising one oxygen or sulfur atom or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl groups are optionally substituted with one or more substituents, independently selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₃₋₆-alkylamino, alkylsulfenyl, alkylsulfinyl, alkylsulfonyl, sulfamoyl, or trifluoromethyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, and amino-C₁₋₆-alkyl.

The term “non-aromatic ring” refers to a group comprising a covalently closed ring that is not aromatic.

The term “alicyclic” refers to a group comprising a non-aromatic ring wherein each of the atoms forming the ring is a carbon atom. Alicyclic rings may be formed by three, four, five, six, seven, eight, nine, or more than nine carbon atoms. In certain embodiments, alicyclics are optionally substituted. In certain embodiments, an alicyclic comprises one or more unsaturated bonds. Alicyclics include cycloalkyls, cycloalkenyls, and cycloalkynyls. Examples of alicyclics include, but are not limited to, cyclopropane, cyclobutane, cyclopentane, cyclopentene, cyclopentadiene, cyclohexane, cyclohexene, 1,3-cyclohexadiene, 1,4-cyclohexadiene, cycloheptane, and cycloheptene. In certain embodiments, alicylcic rings are optionally substituted.

The term “non-aromatic heterocycle” refers to a group comprising a non-aromatic ring wherein one or more atoms forming the ring is a heteroatom. Non-aromatic heterocyclic rings may be formed by three, four, five, six, seven, eight, nine, or more than nine atoms. Non-aromatic heterocycles may be optionally substituted. In certain embodiments, non-aromatic heterocycles comprise one or more carbonyl or thiocarbonyl groups such as, for example, oxo- and thio-containing groups. Examples of non-aromatic heterocycles include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

The term “arylalkyl” refers to a group comprising an aryl group bound to an alkyl group.

The term “ring” refers to any covalently closed structure. Rings include, for example, carbocycles (e.g., aryls and alicyclics), heterocycles (e.g., heteroaryls and non-aromatic heterocycles), aromatics (e.g., aryls and heteroaryls), and non-aromatics (e.g., alicyclics and non-aromatic heterocycles). Rings may be optionally substituted. Rings may form part of a ring system.

The term “ring system” refers to two or more rings, wherein two or more of the rings are fused. The term “fused” refers to structures in which two or more rings share one or more bonds.

The term “null” refers to a group being absent from a structure. For example, in the structure

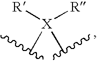

where in certain instances X is N, if X is N, one of R′ or R″ is null, meaning that only three groups are bound to the N.

The term “carboxylic acid bioisostere” refers to a group that is biologically equivalent to a carboxylic acid. For example, carboxylic acid bioisosteres include, but are not limited to, tetrazole, NHSO₂R¹⁵, OC(S)NR¹⁰R¹¹, SC(O)NR¹⁰R¹¹, thiazolidinedione, oxazolidinedione, and 1-oxa-2,4-diazolidine-3,5-dione. In certain embodiments, a carboxylic acid bioisoster comprises the following structure:

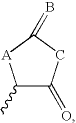

wherein A, B, and C are each independently selected from O, S, and N.

The term “spacer” refers to an atom or group of atoms that separate two or more groups from one another by a desired number of atoms. For example, in certain embodiments, it may be desirable to separate two or more groups by one, two, three, four, five, six, or more than six atoms. In such embodiments, any atom or group of atoms may be used to separate those groups by the desired number of atoms. In certain embodiments, spacers are optionally substituted. In certain embodiments, a spacer comprises an aliphatic. In certain embodiments, a spacer comprises atoms that are part of a ring.

Solely for the purposes of illustration, and without limiting the above definition, some examples of spacers are provided. Examples of 1-atom spacers include, but are not limited to, the following:

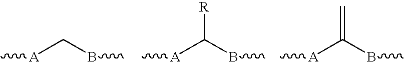

where A and B represent groups which are separated by the desired number of atoms. Examples of 2-atom spacers include, but are not limited to, the following:

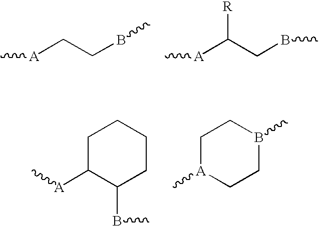

where A and B represent groups which are separated by the desired number of atoms. Examples of 3-atom spacers include, but are not limited to, the following:

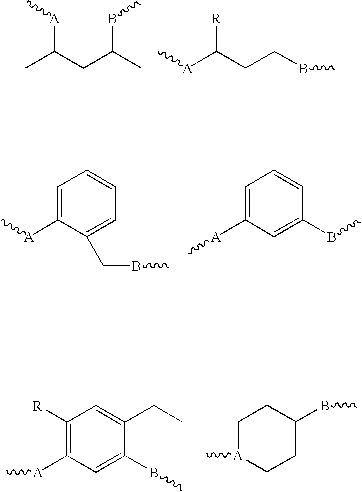

where A and B represent groups that are separated by the desired number of atoms.

In certain embodiments, a spacer separates atoms in a ring. For example, in the structure:

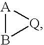

where Q is a 1-atom spacer, the resulting ring is a three-membered ring comprising A, B, and Q, where Q may be optionally substituted. An example of such a structure includes, but is not limited to:

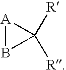

If Q is a 2-atom spacer, then a four-membered ring results; if Q is a three atom spacer, then a five-membered ring results; if Q is a four atom spacer, then a six-membered ring results; if Q is a five atom spacer, then a seven-membered ring results; if Q is a six atom spacer, then an eight-membered ring results; and so on. In certain embodiments, a spacer in a ring comprises a ring, such that the ring formed by the spacer and the ring comprised by the spacer are fused. For example, referring to the structure above where Q is a 3-atom spacer comprising a fused ring includes, but is not limited to, structures such as:

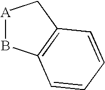

where the fused ring can be fused at any bond of the spacer. Such a fused ring may be optionally substituted and may be heterocyclic or carbocyclic.

As is evident from the above examples, the atoms of a spacer that create the desired separation may themselves be part of a group. That group may be, for example, an aliphatic, heteroaliphatic, haloaliphatic, heterohaloaliphatic, alicyclic, aryl, arylalkyl, heteroaryl, non-aromatic heterocycle, or substituted alkyl all of which are optionally substituted. Thus, the term “1-5 atom spacer” refers to a spacer that separates two groups by 1, 2, 3, 4, or 5 atoms and does not indicate the total size of the group that constitutes the spacer.

The term “linked to form a ring” refers to the circumstance where two atoms that are bound either to a single atom or to atoms that are themselves ultimately bound, are each bound to a linking group, such that the resulting structure forms a ring. That resulting ring comprises the two atoms, the atom (or atoms) that previously linked those atoms, and the linker. For example, if A and B below are “linked to form a ring”

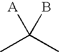

the resulting ring includes A, B, the carbon atom to which both A and B are bound, and a linking group. Unless otherwise indicated, that linking group may be of any length and may be optionally substituted. Referring to the above example, resulting structures include, but are not limited to:

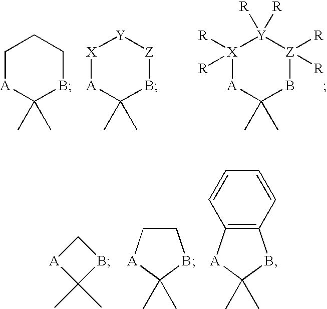

and the like.
In certain embodiments, the two atoms that are linked to form a ring are not bound to the same atom. For example, if A and B, below, are linked to form a ring:

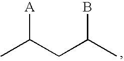

the resulting ring comprises A, B, the 3 carbon atoms that already link A and B, and a linking group. Examples of resulting structures include, but are not limited to:

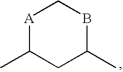

and the like.

The substituent “R” appearing by itself and without a number designation refers to a substituent selected from alkyl, cycloalkyl, aryl, heteroaryl (bonded through a ring carbon) and non-aromatic heterocycle (bonded through a ring carbon).

The term “O-carboxy” refers to a group of formula RC(═O)O—.

The term “C-carboxy” refers to a group of formula —C(═O)OR.

The term “acetyl” refers to a group of formula —C(═O)CH₃.

The term “trihalomethanesulfonyl” refers to a group of formula X₃CS(═O)₂— where X is a halogen.

The term “cyano” refers to a group of formula —CN.

The term “isocyanato” refers to a group of formula —NCO.

The term “thiocyanato” refers to a group of formula —CNS.

The term “isothiocyanato” refers to a group of formula —NCS.

The term “sulfonyl” refers to a group of formula —S(═O)—R.

The term “S-sulfonamido” refers to a group of formula —S(═O)₂NR.

The term “N-sulfonamido” refers to a group of formula RS(═O)₂NH—.

The term “trihalomethanesulfonamido” refers to a group of formula X₃ CS(═O)₂NR—.

The term “O-carbamyl” refers to a group of formula —OC(═O)—NR.

The term “N-carbamyl” refers to a group of formula ROC(═O)NH—.

The term “O-thiocarbamyl” refers to a group of formula —OC(═S)—NR.

The term “N-thiocarbamyl” refers to a group of formula ROC(═S)NH—.

The term “C-amido” refers to a group of formula —C(═O)—NR₂.

The term “N-amido” refers to a group of formula RC(═O)NH—.

The term “ester” refers to a chemical moiety with formula —(R)ₙ—COOR′, where R and R′ are independently selected from alkyl, cycloalkyl, aryl, heteroaryl (bonded through a ring carbon) and non-aromatic heterocycle (bonded through a ring carbon), where n is 0 or 1.

The term “amide” refers to a chemical moiety with formula —(R)ₙ—C(O)NHR′ or —(R)ₙ—NHC(O)R′, where R and R′ are independently selected from alkyl, cycloalkyl, aryl, heteroaryl (bonded through a ring carbon) and heteroalicyclic (bonded through a ring carbon), where n is 0 or 1. In certain embodiments, an amide may be an amino acid or a peptide.

The terms “amine,” “hydroxy,” and “carboxyl” include such groups that have been esterified or amidified. Procedures and specific groups used to achieve esterification and amidification are known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

Unless otherwise indicated, the term “optionally substituted,” refers to a group in which none, one, or more than one of the hydrogen atoms has been replaced with one or more group(s) are independently selected from: alkyl, heteroalkyl, haloalkyl, heteroholoalkyl, cycloalkyl, aryl, arylalkyl, heteroaryl, non-aromatic heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives of amino groups. Such protective derivatives (and protecting groups that may form such protective derivatives) are known to those of skill in the art and may be found in references such as Greene and Wuts, above. In embodiments in which two or more hydrogen atoms have been substituted, the substituent groups may be linked to form a ring.

The term “substantially pure” means an object species (e.g., compound) is the predominant species present (i.e., on a molar basis it is more abundant than any other individual species in the composition). In certain embodiments, a substantially purified fraction is a composition wherein the object species comprises at least about 50 percent (on a molar basis) of all species present. In certain embodiments, a substantially pure composition will comprise more than about 80%, 85%, 90%, 95%, or 99% of all species present in the composition. In certain embodiments, the object species is purified to essential homogeneity (contaminant species cannot be detected in the composition by conventional detection methods) wherein the composition consists essentially of a single species.

The term “tissue-selective” refers to the ability of a compound to modulate a biological activity in one tissue to a greater or lesser degree than it modulates a biological activity in another tissue. The biological activities in the different tissues may be the same or they may be different. The biological activities in the different tissues may be mediated by the same type of target receptor. For example, in certain embodiments, a tissue-selective compound may modulate receptor mediated biological activity in one tissue and fail to modulate, or modulate to a lesser degree, receptor mediated biological activity in another tissue type.

The term “monitoring” refers to observing an effect or absence of any effect. In certain embodiments, one monitors cells after contacting those cells with a compound of the present invention. Examples of effects that may be monitored include, but are not limited to, changes in cell phenotype, cell proliferation, receptor activity, or the interaction between a receptor and a compound known to bind to the receptor.

The term “cell phenotype” refers to physical or biological characteristics of a cell. Examples of characteristics that constitute phenotype included, but are not limited to, cell size, cell proliferation, cell differentiation, cell survival, apoptosis (cell death), or the utilization of a metabolic nutrient (e.g., glucose uptake). Certain changes or the absence of changes in cell phenotype are readily monitored using techniques known in the art.

The term “cell proliferation” refers to the rate at which cells divide. In certain embodiments, cells are in situ in an organism. In certain embodiments, cell are grown in vitro in a vessel. The number of cells growing in a vessel can be quantified by a person skilled in the art (e.g., by counting cells in a defined area using a microscope or by using laboratory apparatus that measure the density of cells in an appropriate medium). One skilled in that art can calculate cell proliferation by determining the number of cells at two or more times.

The term “contacting” refers to bringing two or more materials into close enough proximity that they may interact. In certain embodiments, contacting can be accomplished in a vessel such as a test tube, a petri dish, or the like. In certain embodiments, contacting may be performed in the presence of additional materials. In certain embodiments, contacting may be performed in the presence of cells. In certain of such embodiments, one or more of the materials that are being contacted may be inside a cell. Cells may be alive or may dead. Cells may or may not be intact.

### Certain TPO Compounds

Certain compounds that modulate one or more TPO activity and/or bind to TPO receptors play a role in health. Certain such compounds are useful for treating any of a variety of diseases or conditions.

In certain embodiments, the present invention provides methods of making selective TPO modulators and/or selective TPO receptor binding agents. In certain embodiments, selective TPO modulators are agonists, partial agonists, and/or antagonists for the TPO receptor. In some embodiments, the compounds are described herein or in U.S. application Ser. No. 11/256,572, filed on Oct. 21, 2005 and entitled “THROMBOPOIETIN ACTIVITY MODULATING COMPOUNDS AND METHODS;” WO 03/103686A1, filed Jun. 6, 2003 and entitled “THROMBOPOIETIN MIMETICS;” and WO 01/21180, filed Sep. 22, 2000 and entitled “THROMBOPOIETIN MIMETICS,” each of which is hereby incorporated in its entirety for any reason.

In certain embodiments, the present invention provides compounds useful for making selective TPO modulators and/or selective TPO receptor binding agents. In certain embodiments, selective TPO modulators are agonists, partial agonists, and/or antagonists for the TPO receptor. In certain embodiments, compounds useful for making selective TPO modulators and/or selective TPO receptor binding agents are intermediates in synthetic pathways.

In certain embodiments, the present invention provides methods for making compounds of Formula II, III, or IV:

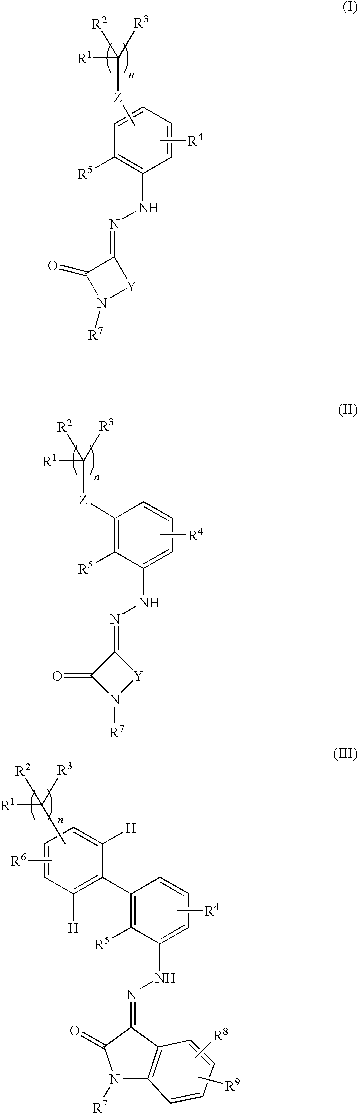

or a pharmaceutically acceptable salt, ester, amide, or prodrug thereof. In certain embodiments, the present invention provides intermediate compounds useful for making compounds of Formula I, II, and/or III.

In certain embodiments, R¹ is selected from hydrogen, CO₂R¹⁰, CONR¹⁰R¹¹, SO₃R¹⁰, and a carboxylic acid bioisostere. In certain embodiments in which R¹ is a carboxylic acid bioisostere, R¹ is selected from tetrazole, NHSO₂R¹⁵, OC(S)NR¹⁰R¹¹, SC(O)NR¹⁰R¹¹, thiazolidinedione, oxazolidinedione, and 1-oxa-2,4-diazolidine-3,5-dione.

In certain embodiments, R² and R³ are each independently selected from hydrogen, OR¹², NR¹²R¹³, an optionally substituted C₁-C₄ aliphatic, an optionally substituted C₁-C₄ haloaliphatic, an optionally substituted C₁-C₄ heteroaliphatic, (CH₂)ₘR¹⁴, an optionally substituted ring, and null. In certain such embodiments, R² and R³ are each independently selected from an optionally substituted C₁-C₄ alkyl, an optionally substituted C₁-C₄ haloalkyl, an optionally substituted C₁-C₄ heteroalkyl. In certain embodiments, R² and R³ taken together form an optionally substituted olefin. In certain embodiments, R² and R³ are linked to form an optionally substituted C₃-C₃ ring. In certain such embodiments, R² and R³ are linked to form an optionally substituted carbocycle, an optionally substituted heterocycle, an optionally substituted aromatic, or an optionally substituted non-aromatic ring. In certain such embodiments, R² and R³ are linked to form an optionally substituted aryl, an optionally substituted heteroaryl, an optionally substituted alicyclic, or an optionally substituted non-aromatic heterocyclic. In certain embodiments, R² and R³ are linked to form an optionally substituted aryl or an optionally substituted heteroaryl. In certain embodiments, R² and R³ are linked to form an optionally substituted aryl. In certain embodiments, R² and R³ are linked to form an aryl.

In certain embodiments, R⁴ is selected from hydrogen, F, Cl, Br, optionally substituted C₁-C₄ aliphatic, optionally substituted C₁-C₄ haloaliphatic, optionally substituted C₁-C₄ heteroaliphatic, and an optionally substituted ring. In certain such embodiments, R⁴ is selected from optionally substituted C₁-C₄ alkyl, optionally substituted C₁-C₄ haloalkyl, and optionally substituted C₁-C₄ heteroalkyl.

In certain embodiments, R⁵ is selected from hydrogen, OR¹⁰, SR¹⁰, NHR¹¹, and CO₂H.

In certain embodiments, R⁶ is selected from hydrogen, OR¹², NR¹²R¹³, F, Cl, Br, optionally substituted C₁-C₄ aliphatic, optionally substituted C₁-C₄ haloaliphatic, optionally substituted C₁-C₄ heteroaliphatic, and an optionally substituted ring. In certain such embodiments, R⁶ is selected from optionally substituted C₁-C₄ alkyl, optionally substituted C₁-C₄ haloalkyl, and optionally substituted C₁-C₄ heteroalkyl. In certain embodiments, R⁶ is selected from an optionally substituted carbocycle, an optionally substituted heterocycle, and optionally substituted aromatic, and an optionally substituted non-aromatic ring. In certain such embodiments, R⁶ is selected from an optionally substituted aryl, an optionally substituted heteroaryl, an optionally substituted alicyclic, and an optionally substituted non-aromatic heterocyclic. In certain embodiments, R⁶ is selected from an optionally substituted aryl and an optionally substituted heteroaryl. In certain embodiments, R⁶ is selected from an optionally substituted aryl. In certain embodiments, R⁶ is an aryl.

In certain embodiments, R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴. In certain such embodiments, R⁷ is selected from an optionally substituted C₁-C₈ alkyl, an optionally substituted C₁-C₈ haloalkyl, an optionally substituted C₁-C₈ heteroalkyl, and an optionally substituted C₁-C₈ heterohaloalkyl. In certain embodiments, R⁷ is selected from an optionally substituted carbocycle, an optionally substituted heterocycle, and optionally substituted aromatic, and an optionally substituted non-aromatic ring. In certain such embodiments, R⁷ is selected from an optionally substituted aryl, an optionally substituted heteroaryl, an optionally substituted alicyclic, and an optionally substituted non-aromatic heterocyclic. In certain embodiments, R⁷ is selected from an optionally substituted aryl and an optionally substituted heteroaryl. In certain embodiments, R⁷ is selected from an optionally substituted aryl. In certain such embodiments, R⁷ is selected from an aryl ring optionally fused to one or more additional rings. In certain embodiments, R⁷ is an aryl. In certain embodiments, R⁷ is an optionally substituted phenyl ring.

In certain embodiments, R⁸ and R⁹ are each independently selected from hydrogen, F, Cl, Br, optionally substituted C₁-C₄ aliphatic, optionally substituted C₁-C₄ haloaliphatic, optionally substituted C₁-C₄ heteroaliphatic, optionally substituted C₁-C₄ heterohaloaliphatic, and an optionally substituted ring. In certain such embodiments, R⁸ and/or R⁹ is independently selected from optionally substituted C₁-C₄ alkyl, optionally substituted C₁-C₄ haloalkyl, optionally substituted C₁-C₄ heteroalkyl, and optionally substituted C₁-C₄ heterohaloalkyl. In certain embodiments, R⁸ and/or R⁹ is selected from an optionally substituted carbocycle, an optionally substituted heterocycle, and optionally substituted aromatic, and an optionally substituted non-aromatic ring. In certain such embodiments, R⁸ and/or R⁹ is selected from an optionally substituted aryl, an optionally substituted heteroaryl, an optionally substituted alicyclic, and an optionally substituted non-aromatic heterocyclic. In certain embodiments, R⁸ and/or R⁹ is selected from an optionally substituted aryl and an optionally substituted heteroaryl. In certain embodiments, R⁸ and/or R⁹ is selected from an optionally substituted aryl. In certain embodiments, R⁸ and/or R⁹ is an aryl.

In certain embodiments, R¹⁰ is selected from hydrogen, a protecting group, optionally substituted C₁-C₄ aliphatic (e.g., methyl), optionally substituted C₁-C₄ haloaliphatic, optionally substituted C₁-C₄ heteroaliphatic (e.g., —CH₂OCH₃), optionally substituted C₁-C₄ heterohaloaliphatic, and an optionally substituted ring. In certain such embodiments, R¹⁰ is selected from optionally substituted C₁-C₄ alkyl, optionally substituted C₁-C₄ haloalkyl, optionally substituted C₁-C₄ heteroalkyl, and optionally substituted C₁-C₄ heterohaloalkyl. In certain embodiments, R¹⁰ is selected from an optionally substituted ring. In certain such embodiments, R¹⁰ is selected from an optionally substituted carbocycle, an optionally substituted heterocycle, and optionally substituted aromatic, and an optionally substituted non-aromatic ring. In certain such embodiments, R¹⁰ is selected from an optionally substituted aryl, an optionally substituted heteroaryl, an optionally substituted alicyclic, and an optionally substituted non-aromatic heterocyclic. In certain embodiments, R¹⁰ is selected from an optionally substituted aryl and an optionally substituted heteroaryl. In certain embodiments, R¹⁰ is selected from an optionally substituted aryl. In certain embodiments, R¹⁰ is an aryl.

In certain embodiments, R¹¹ is selected from hydrogen, SO₂R¹⁵, optionally substituted C₁-C₄ aliphatic, optionally substituted C₁-C₄ haloaliphatic, optionally substituted C₁-C₄ heteroaliphatic, optionally substituted C₁-C₄ heterohaloaliphatic, and an optionally substituted ring. In certain such embodiments, R¹¹ is selected from optionally substituted C₁-C₄ alkyl, optionally substituted C₁-C₄ haloalkyl, optionally substituted C₁-C₄ heteroalkyl, and optionally substituted C₁-C₄ heterohaloalkyl. In certain embodiments, R¹ is selected from an optionally substituted ring. In certain such embodiments, R¹¹ is selected from an optionally substituted carbocycle, an optionally substituted heterocycle, and optionally substituted aromatic, and an optionally substituted non-aromatic ring. In certain such embodiments, R¹¹ is selected from an optionally substituted aryl, an optionally substituted heteroaryl, an optionally substituted alicyclic, and an optionally substituted non-aromatic heterocyclic. In certain embodiments, R¹¹ is selected from an optionally substituted aryl and an optionally substituted heteroaryl. In certain embodiments, R¹¹ is selected from an optionally substituted aryl. In certain embodiments, R¹¹ is an aryl.

In some embodiments, R¹² and R¹³ are each independently selected from hydrogen, optionally substituted C₁-C₄ aliphatic, optionally substituted C₁-C₄ haloaliphatic, optionally substituted C₁-C₄ heteroaliphatic, optionally substituted C₁-C₄ heterohaloaliphatic, an optionally substituted ring, and (CH₂)ₘR¹⁴. In certain such embodiments, R¹² and/or R¹³ is independently selected from optionally substituted C₁-C₄ alkyl, optionally substituted C₁-C₄ haloalkyl, optionally substituted C₁-C₄ heteroalkyl, and optionally substituted C₁-C₄ heterohaloalkyl. In certain embodiments, R¹² and/or R¹³ is selected from an optionally substituted carbocycle, an optionally substituted heterocycle, and optionally substituted aromatic, and an optionally substituted non-aromatic ring. In certain such embodiments, R¹² and/or R¹³ is selected from an optionally substituted aryl, an optionally substituted heteroaryl, an optionally substituted alicyclic, and an optionally substituted non-aromatic heterocyclic. In certain embodiments, R¹² and/or R¹³ is selected from an optionally substituted aryl and an optionally substituted heteroaryl. In certain embodiments, R¹² and/or R¹³ is selected from an optionally substituted aryl. In certain embodiments, R¹² and/or R¹³ is an aryl. In certain embodiments, one of R¹² or R¹³ is a ring and the other of R¹² and R¹³ is hydrogen.

In certain embodiments, R¹² and R¹³ are linked to form an optionally substituted C₂-C₈ heterocycle. In certain embodiments, R¹² and R¹³ are linked to form an optionally substituted C₂-C₈ heteroaryl. In certain embodiments, R¹² and R¹³ are linked to form an optionally substituted C₂-C₈ non-aromatic heterocycle.

In certain embodiments, R¹⁴ is selected from an optionally substituted ring. In certain such embodiments, R¹⁴ is selected from an optionally substituted carbocycle, an optionally substituted heterocycle, and optionally substituted aromatic, and an optionally substituted non-aromatic ring. In certain such embodiments, R¹⁴ is selected from an optionally substituted aryl, an optionally substituted heteroaryl, an optionally substituted alicyclic, and an optionally substituted non-aromatic heterocyclic. In certain embodiments, R¹⁴ is selected from an optionally substituted aryl and an optionally substituted heteroaryl. In certain embodiments, R¹⁴ is selected from an optionally substituted aryl. In certain embodiments, R¹⁴ is an aryl.

In certain embodiments, R¹⁵ is selected from hydrogen, optionally substituted C₁-C₃ aliphatic, optionally substituted C₁-C₃ haloaliphatic, and optionally substituted ring. In certain such embodiments, R¹⁵ is selected from optionally substituted C₁-C₃ alkyl, and optionally substituted C₁-C₃ haloalkyl. In certain embodiments, R¹⁵ is an optionally substituted aryl. In certain embodiments, R¹⁵ is selected from an alkyl, a haloalkyl, an alicyclic, and an aryl. In certain embodiments, R¹⁵ is selected from an optionally substituted ring. In certain such embodiments, R¹⁵ is selected from an optionally substituted carbocycle, an optionally substituted heterocycle, and optionally substituted aromatic, and an optionally substituted non-aromatic ring. In certain such embodiments, R¹⁵ is selected from an optionally substituted aryl, an optionally substituted heteroaryl, an optionally substituted alicyclic, and an optionally substituted non-aromatic heterocyclic. In certain embodiments, R¹⁵ is selected from an optionally substituted aryl and an optionally substituted heteroaryl. In certain embodiments, R¹⁵ is selected from an optionally substituted aryl. In certain embodiments, R¹⁵ is an aryl.

In certain embodiments, Y is a 1, 2, 3, 4, 5, 7, or 8 atom spacer. In certain embodiments, Y is a 1-4 atom spacer selected from optionally substituted C₁-C₆ aliphatic and optionally substituted C₁-C₆ heteroaliphatic. In certain such embodiments, Y is a 1-4 atom spacer selected from optionally substituted C₁-C₆ alkyl, optionally substituted C₁-C₆ heteroalkyl, optionally substituted C₂-C₆ alkenyl, and optionally substituted C₂-C₆ heteroalkenyl.

In certain embodiments, Y is a 1-4 atom spacer comprising a ring. In certain such embodiments, Y is selected from optionally substituted phenyl, optionally substituted monocyclic heteroaryl, optionally substituted C₃-C₅ heterocycle, and optionally substituted alicyclic, including, but not limited to, optionally substituted cycloalkyl and optionally substituted cycloalkenyl.

In certain embodiments, Y is a 2-6 atom spacer comprising both (1) a ring selected from optionally substituted phenyl, optionally substituted monocyclic heteroaryl, optionally substituted C₃-C₅ heterocycle, and optionally substituted alicyclic and (2) 1-4 atoms selected from optionally substituted C₁-C₆ aliphatic, and optionally substituted C₁-C₆ heteroaliphatic.

In certain embodiments, Y is not —N═CR⁶— orientated to form the dihydropyrazole. Thus, in such embodiments, the ring that includes Y cannot be:

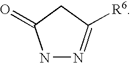

In certain embodiments, Y is selected from:

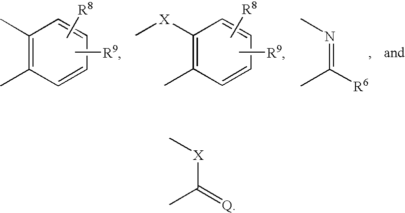

In certain embodiments, Q is selected from O and S.

In certain embodiments, X is selected from O, S, NR¹⁰, and CR¹⁰R¹⁰;

In certain embodiments, Z is a 1 to 5 atom spacer. In certain embodiments, Z is a 2-5 atom spacer selected from an optionally substituted C₆-C₁₀ aryl and an optionally substituted C₁-C₃ heteroaryl. In certain embodiments, Z is a 1-5 atom spacer selected from an optionally substituted C₁-C₆ alkyl, an optionally substituted C₁-C₆ heteroalkyl, an optionally substituted C₁-C₆ haloalkyl, an optionally substituted C₂-C₆ alkenyl, an optionally substituted C₂-C₆ heteroalkenyl, an optionally substituted C₂-C₆ haloalkenyl, an optionally substituted C₂-C₆ alkynyl, and an optionally substituted C₂-C₆ heteroalkyl.

In certain embodiments, m is 0, 1, or 2.

In certain embodiments, n is 0 or 1. In embodiments in which n is 0, R¹ binds directly to Z and R² and/or R³ are null, as appropriate. For example, if Z is a phenyl ring and n is 0, then R¹ binds directly to the phenyl ring and both R¹ and R² are null.

In embodiments in which two or more of a particular group are present, the identities of those two or more particular groups are selected independently and, thus, may be the same or different from one another. For example, certain compounds of the invention comprise two or more R¹⁴ groups. The identities of those two or more R¹⁴ groups are each selected independently. Thus, in certain embodiments, those R¹⁴ groups are all the same as one another; in certain embodiments, those R¹⁴ groups are all different from one another; and in certain embodiments, some of those R¹⁴ groups are the same as one another and some are different from one another. This independent selection applies to any group that is present in a compound more than once.

One of ordinary skill in the art will recognize that the complete lists of possible identities for each above-listed group (all R groups, Y, Q, Z, m, and n) may be narrowed to provide shorter lists of possible identities. For example, since in certain embodiments R¹ is selected from hydrogen, CO₂R¹⁰, CONR¹⁰R¹¹, SO₃R¹⁰, and a carboxylic acid bioisostere, it is to be understood that in certain embodiments, R¹ may be selected from CO₂R¹¹, CONR¹⁰R¹¹, and SO₃R¹⁰, because each of those possible identities is included on the longer list of possible identities. One of ordinary skill in the art will also recognize that broader terms include combinations of narrower terms, which may be substituted and selected. For example, in certain embodiments, R² is selected from an optionally substituted C₁-C₄ aliphatic. Because aliphatics include, but are not limited to, alkyls and alkenes, in certain embodiments, R² may be selected from an optionally substituted C₁-C₄ alkyl and an optionally substituted C₁-C₄ alkenyl. Similarly, in certain embodiments, R² is selected from an optionally substituted C₂-C₃ alkyl and an optionally substituted C₂-C₄ alkenyl, because those alkyls and alkenyls are included in the definition of C₁-C₄ aliphatics.

One of ordinary skill in the art will also understand that the above listed groups may be selected in any combination. For example, in certain embodiments, R¹ is selected from hydrogen, CO₂R¹⁰, CONR¹⁰R¹¹, SO₃R¹⁰, and a carboxylic acid bioisostere; and R² is selected from hydrogen, 0R¹², NR¹²R¹³, an optionally substituted C₁-C₄ aliphatic, an optionally substituted C₁-C₄ haloaliphatic, an optionally substituted C₁-C₄ heteroaliphatic, (CH₂)ₘR¹⁴, an optionally substituted ring, and null. Therefore, in certain embodiments, R¹ may be selected from hydrogen, and CO₂R¹⁰; and at the same time R² may be selected from hydrogen, OR¹², NR¹²R¹³, and an optionally substituted C₁-C₄ aliphatic, because those lists of possible identities are included within the previous lists of possible identities. Such selection of combinations are included for all groups herein.

In certain embodiments, a compound of Formula I, II, or III is a selective TPO modulator. In certain embodiments, a compound of Formula I, II, or III is a selective TPO receptor agonist. In certain embodiments, a compound of Formula I, II, or III is a selective TPO receptor antagonist. In certain embodiments, a compound of Formula I, II, or III is a selective TPO receptor partial agonist. In certain embodiments, a compound of Formula I, II, or III is a tissue-specific selective TPO modulator. In certain embodiments, a compound of Formula I, II, or III is a selective TPO receptor binding compound. In certain embodiments, a compound of Formula I, II, or III is a TPO mimic.

In certain embodiments, the present invention provides methods of making compounds including, but not limited to:
3′-{N′-[1-(3,4-Dimethyl-phenyl)-2-oxo-1,2-dihydro-indol-3-ylidene]-hydrazino}-2′-hydroxy-biphenyl-3-carboxylic acid (Compound 101);
2′-Hydroxy-3′-{N′-[2-oxo-1-(4-propyl-phenyl)-1,2-dihydro-indol-3-ylidene]-hydrazino}-biphenyl-3-carboxylic acid (Compound 102);
2′-Hydroxy-3′-{N′-[2-oxo-1-(4-ethyl-phenyl)-1,2-dihydro-indol-3-ylidene]-hydrazino}-biphenyl-3-carboxylic acid (Compound 103);
2′-Hydroxy-3′-{N′-[2-oxo-1-(4-trifluoromethoxy-phenyl)-1,2-dihydro-indol-3-ylidene]-hydrazino}-biphenyl-3-carboxylic acid (Compound 104);
3′-{N′-[1-(3-Fluoro-4-methoxy-phenyl)-2-oxo-1,2-dihydro-indol-3-ylidene]-hydrazino}-2′-hydroxy-biphenyl-3-carboxylic acid (Compound 105);
3′-{N′-[1-(3,4-Dimethyl-phenyl)-2-oxo-1,2-dihydro-indol-3-ylidene]-hydrazino}-2′-hydroxy-biphenyl-4-carboxylic acid (Compound 106);
2′-Hydroxy-3′-{N′-[2-oxo-1-(4-propyl-phenyl)-1,2-dihydro-indol-3-ylidene]-hydrazino}-biphenyl-4-carboxylic acid (Compound 107);
2′-Hydroxy-3′-{N′-[2-oxo-1-(4-ethyl-phenyl)-1,2-dihydro-indol-3-ylidene]-hydrazino}-biphenyl-4-carboxylic acid (Compound 108);
3′-{N′-[4-tert-Butyl-phenyl)-2-oxo-1,2-dihydro-indol-3-ylidene]-hydrazino}-2′-hydroxy-biphenyl-3-carboxylic acid (Compound 109);
2′-Hydroxy-3′-{N′-[2-oxo-1-(4-trifluoromethyl-phenyl)-1,2-dihydro-indol-3-ylidene]-hydrazino}-biphenyl-3-carboxylic acid (Compound 110);
3′-[N′-(1-Benzyl-5-chloro-2-oxo-1,2-dihydro-indol-3-ylidene)-hydrazino]-2′-hydroxy-biphenyl-3-carboxylic acid (Compound 111);
3′-[N′-(1-Benzyl-5-methyl-2-oxo-1,2-dihydro-indol-3-ylidene)-hydrazino]-2′-hydroxy-biphenyl-3-carboxylic acid (Compound 112);
3′-[N′-(1-Benzyl-2-oxo-1,2-dihydro-indol-3-ylidene)-hydrazino]-2′-hydroxy-biphenyl-3-carboxylic acid (Compound 113);
2′-Hydroxy-3′-{N′-[2-oxo-1-(4-trifluoromethyl-phenyl)-1,2-dihydro-indol-3-ylidene]-hydrazino}-biphenyl-4-carboxylic acid (Compound 114);
3′-{N′-[1-(3,4-Dichloro-phenyl)-2-oxo-1,2-dihydro-indol-3-ylidene]-hydrazino}-2′-hydroxy-biphenyl-3-carboxylic acid (Compound 115);
2′-Hydroxy-3′-{N′-[1-(4-methyl-3-trifluoromethyl-phenyl)-2-oxo-1,2-dihydro-indol-3-ylidene]-hydrazino}-biphenyl-3-carboxylic acid (Compound 116);
3′-{N′-[1-(3-Fluoro-4-trifluoromethyl-phenyl)-2-oxo-1,2-dihydro-indol-3-ylidene]-hydrazino}-2′-hydroxy-biphenyl-3-carboxylic acid (Compound 117);
3′-{N′-[1-(3,5-Bis-trifluoromethyl-phenyl)-2-oxo-1,2-dihydro-indol-3-ylidene]-hydrazino}-2′-hydroxy-biphenyl-3-carboxylic acid (Compound 118);
3′-{N′-[3-(3,4-Dimethyl-phenyl)-4-oxo-2-thioxo-thiazolidin-5-ylidene]-hydrazino}-2′-hydroxy-biphenyl-3-carboxylic acid (Compound 119);
2′-Hydroxy-3′-{N′-[1-(4-isopropyl-phenyl)-2-oxo-1,2-dihydro-indol-3-ylidene]-hydrazino}-biphenyl-3-carboxylic acid (Compound 120);
3′-{N′-[1-(2-Fluoro-4-trifluoromethyl-phenyl)-2-oxo-1,2-dihydro-indol-3-ylidene]-hydrazino}-2′-hydroxy-biphenyl-3-carboxylic acid (Compound 121);
3′-{N′-[1-(2-Fluoro-4-methyl-phenyl)-2-oxo-1,2-dihydro-indol-3-ylidene]-hydrazino}-2′-hydroxy-biphenyl-3-carboxylic acid (Compound 122);
3′-{N′-[1-(4-Chloro-3-trifluoromethyl-phenyl)-2-oxo-1,2-dihydro-indol-3-ylidene]-hydrazino}-2′-hydroxy-biphenyl-3-carboxylic acid (Compound 123);
3′-{N′-[1-(4-Butyl-phenyl)-2-oxo-1,2-dihydro-indol-3-ylidene]-hydrazino}-2′-hydroxy-biphenyl-3-carboxylic acid (Compound 124);
3′-{N′-[1-(3-Fluoro-phenyl)-2-oxo-1,2-dihydro-indol-3-ylidene]-hydrazino}-2′-hydroxy-biphenyl-3-carboxylic acid (Compound 125);
2′-Hydroxy-3′-[N′-(2-oxo-1-m-tolyl-1,2-dihydro-indol-3-ylidene)-hydrazino]-biphenyl-3-carboxylic acid (Compound 126);
3′-{N′-[1-(4-Fluoro-phenyl)-2-oxo-1,2-dihydro-indol-3-ylidene]-hydrazino}-2′-hydroxy-biphenyl-3-carboxylic acid (Compound 127);
3′-[N′-(1-Benzyl-5-methoxy-2-oxo-1,2-dihydro-indol-3-ylidene)-hydrazino]-2′-hydroxy-biphenyl-3-carboxylic acid (Compound 128);
2′-Hydroxy-3′-{N′-[2-oxo-1-(3-trifluoromethyl-phenyl)-1,2-dihydro-indol-3-ylidene]-hydrazino}-biphenyl-3-carboxylic acid (Compound 129);
3′-{N′-[5-Chloro-1-(4-isopropyl-phenyl)-2-oxo-1,2-dihydro-indol-3-ylidene]-hydrazino}-2′-hydroxy-biphenyl-3-carboxylic acid (Compound 130);
3′-{N′-[6-Chloro-1-(4-isopropyl-phenyl)-2-oxo-1,2-dihydro-indol-3-ylidene]-hydrazino}-2′-hydroxy-biphenyl-3-carboxylic acid (Compound 131);
3′-{N′-[5-Fluoro-1-(4-isopropyl-phenyl)-2-oxo-1,2-dihydro-indol-3-ylidene]-hydrazino}-2′-hydroxy-biphenyl-3-carboxylic acid (Compound 132);
3′-{N′-[5-Methoxy-1-(4-isopropyl-phenyl)-2-oxo-1,2-dihydro-indol-3-ylidene]-hydrazino}-2′-hydroxy-biphenyl-3-carboxylic acid (Compound 133);
3′-{N′-[1-(3,4-Dimethyl-phenyl)-5-fluoro-2-oxo-1,2-dihydro-indol-3-ylidene]-hydrazino}-2′-hydroxy-biphenyl-3-carboxylic acid (Compound 134);
3′-{N′-[1-(4-Fluoro-3-trifluoromethyl-phenyl)-5-fluoro-2-oxo-1,2-dihydro-indol-3-ylidene]-hydrazino}-2′-hydroxy-biphenyl-3-carboxylic acid (Compound 135);
3′-{N′-[1-(3,5-Dichloro-phenyl)-5-fluoro-2-oxo-1,2-dihydro-indol-3-ylidene]-hydrazino}-2′-hydroxy-biphenyl-3-carboxylic acid (Compound 136);
3′-{N′-[1-(4-Propyl-phenyl)-6-chloro-2-oxo-1,2-dihydro-indol-3-ylidene]-hydrazino}-2′-hydroxy-biphenyl-3-carboxylic acid (Compound 137);
(±)-2′-Hydroxy-3′-(N′-{2-oxo-1-[4-(2,2,2-trifluoro-1-hydroxy-ethyl)-phenyl]-1,2-dihydro-indol-3-ylidene}-hydrazino)-biphenyl-3-carboxylic acid (Compound 138);
(±)-2′-Hydroxy-3′-(N′-{2-oxo-1-[4-(2,2,2-trifluoro-1-methoxy-ethyl)-phenyl]-1,2-dihydro-indol-3-ylidene}-hydrazino)-biphenyl-3-carboxylic acid (Compound 139);
2′-Hydroxy-3′-(N′-{2-oxo-1-[4-(2,2,2-trifluoro-ethyl)-phenyl]-1,2-dihydro-indol-3-ylidene}-hydrazino)-biphenyl-3-carboxylic acid (Compound 140);
3′-{N′-[1-(3,4-Dimethyl-phenyl)-4,5-dimethyl-2-oxo-1,2-dihydro-indol-3-ylidene]-hydrazino}-2′-hydroxy-biphenyl-3-carboxylic acid (Compound 141);
3′-{N′-[1-(3,4-Dimethyl-phenyl)-5-fluoro-4-methyl-2-oxo-1,2-dihydro-indol-3-ylidene]-hydrazino}-2′-hydroxy-biphenyl-3-carboxylic acid (Compound 142);
3′-{N′-[1-(3,4-Dimethyl-phenyl)-5-fluoro-6-methyl-2-oxo-1,2-dihydro-indol-3-ylidene]-hydrazino}-2′-hydroxy-biphenyl-3-carboxylic acid (Compound 143);
5-(4-{N′-[1-(3,4-Dimethyl-phenyl)-2-oxo-6-trifluoromethyl-1,2-dihydro-indol-3-ylidene]-hydrazino}-3-hydroxy-benzylidene)-thiazolidine-2,4-dione (Compound 144);
5-(4-{N′-[1-(3,4-Dimethyl-phenyl)-2-oxo-1,2-dihydro-indol-3-ylidene]-hydrazino}-3-hydroxy-benzylidene)-thiazolidine-2,4-dione (Compound 145);
3′-{N′-[1-(3,4-Dimethyl-phenyl)-4-fluoro-2-oxo-6-trifluoromethyl-1,2-dihydro-indol-3-ylidene]-hydrazino}-2′-hydroxy-biphenyl-3-carboxylic acid (Compound 146);
3′-{N′-[4-Chloro-1-(3,4-dimethyl-phenyl)-2-oxo-6-trifluoromethyl-1,2-dihydro-indol-3-ylidene]-hydrazino}-2′-hydroxy-biphenyl-3-carboxylic acid (Compound 147);
5-(4-{N′-[1-(3,4-Dimethyl-phenyl)-4-fluoro-2-oxo-6-trifluoromethyl-1,2-dihydro-indol-3-ylidene]-hydrazino}-3-hydroxy-benzylidene)-thiazolidine-2,4-dione (Compound 148);
5-(4-{N′-[4-Chloro-1-(3,4-dimethyl-phenyl)-2-oxo-6-trifluoromethyl-1,2-dihydro-indol-3-ylidene]-hydrazino}-3-hydroxy-benzylidene)-thiazolidine-2,4-dione (Compound 149);
3-(4-{N′-[1-(3,4-Dimethyl-phenyl)-2-oxo-6-trifluoromethyl-1,2-dihydro-indol-3-ylidene]-hydrazino}-3-hydroxy-phenyl)-acrylic acid (Compound 150);
1-(3,4-Dimethyl-phenyl)-3-{[2-hydroxy-4-(4-oxo-2-thioxo-thiazolidin-5-ylidenemethyl)-phenyl]-hydrazono}-6-trifluoromethyl-1,3-dihydro-indol-2-one (Compound 151);
1-(3,4-Dimethyl-phenyl)-4-fluoro-3-{[2-hydroxy-4-(4-oxo-2-thioxo-thiazolidin-5-ylidenemethyl)-phenyl]-hydrazono}-6-trifluoromethyl-1,3-dihydro-indol-2-one (Compound 152);
5-(3-{N′-[1-(3,4-Dimethyl-phenyl)-2-oxo-6-trifluoromethyl-1,2-dihydro-indol-3-ylidene]-hydrazino}-2-hydroxy-benzylidene)-thiazolidine-2,4-dione (Compound 153);
3′-{N′-[5-Chloro-2-oxo-1-(4-propyl-phenyl)-1,2-dihydro-indol-3-ylidene]-hydrazino}-2′-hydroxy-biphenyl-3-carboxylic acid (Compound 154);
2′-Hydroxy-3′-{N′-[1-(4-methylsulfanyl-phenyl)-2-oxo-1,2-dihydro-indol-3-ylidene]-hydrazino}-biphenyl-3-carboxylic acid (Compound 155);
2′-Hydroxy-3′-{N′-[1-(4-methoxymethyl-phenyl)-2-oxo-1,2-dihydro-indol-3-ylidene]-hydrazino}-biphenyl-3-carboxylic acid (Compound 156);
(±)-2′-Hydroxy-3′-(N′-{2-oxo-1-[4-(2,2,2-trifluoro-1-hydroxy-1-methyl-ethyl)-phenyl]-1,2-dihydro-indol-3-ylidene}-hydrazino)-biphenyl-3-carboxylic acid (Compound 157);
3′-{N′-[5-Fluoro-1-(4-methyl-3-trifluoromethyl-phenyl)-2-oxo-1,2-dihydro-indol-3-ylidene]-hydrazino}-2′-hydroxy-biphenyl-3-carboxylic acid (Compound 158);
2′-Hydroxy-3′-(N′-{2-oxo-1-[4-(2,2,2-trifluoro-1-methoxy-1-methyl-ethyl)-phenyl]-1,2-dihydro-indol-3-ylidene}-hydrazino)-biphenyl-3-carboxylic acid (Compound 159);
3′-{N′-[1-(3,4-Dimethyl-phenyl)-6-fluoro-2-oxo-1,2-dihydro-indol-3-ylidene]-hydrazino}-2′-hydroxy-biphenyl-3-carboxylic acid (Compound 160);
3′-{N′-[6-Fluoro-1-(4-isopropyl-phenyl)-2-oxo-1,2-dihydro-indol-3-ylidene]-hydrazino}-2′-hydroxy-biphenyl-3-carboxylic acid (Compound 161);
3′-{N′-[1-(3,4-Dimethyl-phenyl)-2-oxo-5-trifluoromethyl-1,2-dihydro-indol-3-ylidene]-hydrazino}-2′-hydroxy-biphenyl-3-carboxylic acid (Compound 162);
3′-{N′-[6-Fluoro-2-oxo-1-(4-propyl-phenyl)-1,2-dihydro-indol-3-ylidene]-hydrazino}-2′-hydroxy-biphenyl-3-carboxylic acid (Compound 163);
2′-Hydroxy-3′-{N′-[2-oxo-1-(4-propyl-phenyl)-5-trifluoromethyl-1,2-dihydro-indol-3-ylidene]-hydrazino}-biphenyl-3-carboxylic acid (Compound 164);
3′-{N′-[4,5-Difluoro-1-(4-isopropyl-phenyl)-2-oxo-1,2-dihydro-indol-3-ylidene]-hydrazino}-2′-hydroxy-biphenyl-3-carboxylic acid (Compound 165);
2′-Hydroxy-3′-[N′-(2-oxo-1-piperidin-4-yl-1,2-dihydro-indol-3-ylidene)-hydrazino]-biphenyl-3-carboxylic acid (Compound 166);
3′-{N′-[5-Fluoro-1-(2-fluoro-4-methyl-phenyl)-2-oxo-1,2-dihydro-indol-3-ylidene]-hydrazino}-2′-hydroxy-biphenyl-3-carboxylic acid (Compound 167);
2′-Hydroxy-3′-[N′-(1-methyl-2-oxo-1,2-dihydro-indol-3-ylidene)-hydrazino]-biphenyl-3-carboxylic acid (Compound 168);
3′-[N′-(1-Cyclopentyl-2-oxo-1,2-dihydro-indol-3-ylidene)-hydrazino]-2′-hydroxy-biphenyl-3-carboxylic acid (Compound 169);
3′-{N′-[1-(3,4-Dimethyl-phenyl)-6-methyl-2-oxo-1,2-dihydro-indol-3-ylidene]-hydrazino}-2′-hydroxy-biphenyl-3-carboxylic acid (Compound 170);
2′-Hydroxy-3′-[N′-(2-oxo-1-phenyl-1,2-dihydro-indol-3-ylidene)-hydrazino]-biphenyl-3-carboxylic acid (Compound 171);
3′-[N′-(6-Fluoro-2-oxo-1-phenyl-2,3-dihydro-1H-indol-3-yl)-hydrazino]-2′-hydroxy-biphenyl-3-carboxylic acid (Compound 172);
3′-{N′-[1-(3,4-Dimethyl-phenyl)-6-isopropyl-2-oxo-1,2-dihydro-indol-3-ylidene]-hydrazino}-2′-hydroxy-biphenyl-3-carboxylic acid (Compound 173);
3′-{N′-[1-(3,4-Dimethyl-phenyl)-4-isopropyl-2-oxo-1,2-dihydro-indol-3-ylidene]-hydrazino}-2′-hydroxy-biphenyl-3-carboxylic acid (Compound 174);
3′-{N′-[1-(3,4-Dimethyl-phenyl)-2-oxo-6-trifluoromethyl-1,2-dihydro-indol-3-ylidene]-hydrazino}-4-fluoro-2′-hydroxy-biphenyl-3-carboxylic acid (Compound 175);
5′-Chloro-3′-{N′-[1-(3,4-dimethyl-phenyl)-2-oxo-6-trifluoromethyl-1,2-dihydro-indol-3-ylidene]-hydrazino}-4-fluoro-2′-hydroxy-biphenyl-3-carboxylic acid (Compound 176);
3′-{N′-[1-(3,4-Dimethyl-phenyl)-2-oxo-6-trifluoromethyl-1,2-dihydro-indol-3-ylidene]-hydrazino}-6-fluoro-2′-hydroxy-biphenyl-3-carboxylic acid (Compound 177);
3′-{N′-[1-(3,4-Dimethyl-phenyl)-4,5-difluoro-2-oxo-1,2-dihydro-indol-3-ylidene]-hydrazino}-2′-hydroxy-biphenyl-3-carboxylic acid (Compound 178);
3′-{N′-[1-(3,4-Dimethyl-phenyl)-2-oxo-6-trifluoromethyl-1,2-dihydro-indol-3-ylidene]-hydrazino}-2′-hydroxy-3-methyl-biphenyl-4-carboxylic acid (Compound 179);
3′-{N′-[1-(3,4-Dimethyl-phenyl)-2-oxo-6-trifluoromethyl-2,3-dihydro-1H-indol-3-yl]-hydrazino}-2-fluoro-2′-hydroxy-biphenyl-4-carboxylic acid (Compound 180);
3′-{N′-[1-(3,4-Dimethyl-phenyl)-4-fluoro-2-oxo-6-trifluoromethyl-2,3-dihydro-1H-indol-3-yl]-hydrazino}-4-fluoro-2′-hydroxy-biphenyl-3-carboxylic acid (Compound 181);
5′-Chloro-3′-{N′-[1-(3,4-dimethyl-phenyl)-4-fluoro-2-oxo-6-trifluoromethyl-1,2-dihydro-indol-3-ylidene]-hydrazino}-4-fluoro-2′-hydroxy-biphenyl-3-carboxylic acid (Compound 182);
3-[(3′-Carboxy-2-hydroxy-biphenyl-3-yl)-hydrazono]-1-(3,5-dimethyl-phenyl)-2-oxo-2,3-dihydro-1H-indole-6-carboxylic acid methyl ester (Compound 183);
3-[(3′-Carboxy-4′-fluoro-2-hydroxy-biphenyl-3-yl)-hydrazono]-1-(3,5-dimethyl-phenyl)-2-oxo-2,3-dihydro-1H-indole-6-carboxylic acid methyl ester (Compound 184);
3-[(3′-Carboxy-4′-fluoro-2-hydroxy-biphenyl-3-yl)-hydrazono]-1-(3,4-dimethyl-phenyl)-2-oxo-2,3-dihydro-1H-indole-6-carboxylic acid methyl ester (Compound 185);
3-[(3′-Carboxy-5-chloro-4′-fluoro-2-hydroxy-biphenyl-3-yl)-hydrazono]-1-(3,5-dimethyl-phenyl)-2-oxo-2,3-dihydro-1H-indole-6-carboxylic acid methyl ester (Compound 186);
3′-{N′-[1-(2-Cyano-thiophen-3-yl)-2-oxo-1,2-dihydro-indol-3-ylidene]-hydrazino}-2′-hydroxy-biphenyl-3-carboxylic acid (Compound 187);
2′-Hydroxy-3′-[N′-(2-oxo-1-thiophen-3-yl-1,2-dihydro-indol-3-ylidene)-hydrazino]-biphenyl-3-carboxylic acid (Compound 188);
3-[(3′-Carboxy-2-hydroxy-biphenyl-3-yl)-hydrazono]-1-(3,4-dimethyl-phenyl)-2-oxo-2,3-dihydro-1H-indole-6-carboxylic acid methyl ester (Compound 189);
3′-{N′-[1-(4-Chloro-3-trifluoromethyl-phenyl)-6-cyano-2-oxo-1,2-dihydro-indol-3-ylidene]-hydrazino}-2′-hydroxy-biphenyl-3-carboxylic acid (Compound 190);
5′-Chloro-3′-{N′-[6-cyano-1-(4-isopropyl-phenyl)-2-oxo-1,2-dihydro-indol-3-ylidene]-hydrazino}-4-fluoro-2′-hydroxy-biphenyl-3-carboxylic acid (Compound 191);
3′-{N′-[6-Cyano-1-(4-isopropyl-phenyl)-2-oxo-1,2-dihydro-indol-3-ylidene]-hydrazino}-4-fluoro-2′-hydroxy-biphenyl-3-carboxylic acid (Compound 192);
(±)-1-(3,4-Dimethyl-phenyl)-3-{[2-hydroxy-3′-(2,2,2-trifluoro-1-hydroxy-ethyl)-biphenyl-3-yl]-hydrazono}-6-methanesulfonyl-1,3-dihydro-indol-2-one (Compound 193);
3′-{N′-[6-Cyano-1-(4-isopropyl-phenyl)-2-oxo-1,2-dihydro-indol-3-ylidene]-hydrazino}-2′-hydroxy-biphenyl-3-carboxylic acid (Compound 194);
3′-{N′-[1-(3,4-Dimethyl-phenyl)-5-nitro-2-oxo-1,2-dihydro-indol-3-ylidene]-hydrazino}-2′-hydroxy-biphenyl-3-carboxylic acid (Compound 195);
3′-{N′-[1-(3,4-Dimethyl-phenyl)-6-methanesulfonyl-2-oxo-1,2-dihydro-indol-3-ylidene]-hydrazino}-2′-hydroxy-biphenyl-3-carboxylic acid (Compound 196);
3′-{N′-[6-Cyano-1-(3,4-dimethyl-phenyl)-2-oxo-1,2-dihydro-indol-3-ylidene]-hydrazino}-2′-hydroxy-biphenyl-3-carboxylic acid (Compound 197);
3′-{N′-[1-(5-Cyano-pyridin-3-yl)-2-oxo-1,2-dihydro-indol-3-ylidene]-hydrazino}-2′-hydroxy-biphenyl-3-carboxylic acid (Compound 198);
3′-[N′-(1-Furan-3-yl-2-oxo-1,2-dihydro-indol-3-ylidene)-hydrazino]-2′-hydroxy-biphenyl-3-carboxylic acid (Compound 199);
3′-[N′-(1-Benzo[1,3]dioxol-5-yl-2-oxo-1,2-dihydro-indol-3-ylidene)-hydrazino]-2′-hydroxy-biphenyl-3-carboxylic acid (Compound 200);
2′-Hydroxy-3′-{N′-[1-(3-methyl-thiophen-2-yl)-2-oxo-1,2-dihydro-indol-3-ylidene]-hydrazino}-biphenyl-3-carboxylic acid (Compound 201);
2′-Hydroxy-3′-[N′-(2-oxo-1-thiophen-2-yl-1,2-dihydro-indol-3-ylidene)-hydrazino]-biphenyl-3-carboxylic acid (Compound 202);
2′-Hydroxy-3′-{N′-[1-(4-isopropyl-phenyl)-2-oxo-6-trifluoromethyl-1,2-dihydro-indol-3-ylidene]-hydrazino}-biphenyl-3-carboxylic acid (Compound 203);
2′-Hydroxy-3′-{N′-[2-oxo-1-(4-propyl-phenyl)-6-trifluoromethyl-1,2-dihydro-indol-3-ylidene]-hydrazino}-biphenyl-3-carboxylic acid (Compound 204);
3′-{N′-[1-(4-Ethyl-phenyl)-5,7-difluoro-2-oxo-1,2-dihydro-indol-3-ylidene]-hydrazino}-2′-hydroxy-biphenyl-3-carboxylic acid (Compound 205);
3′-{N′-[1-(3,4-Dimethyl-phenyl)-5,7-difluoro-2-oxo-1,2-dihydro-indol-3-ylidene]-hydrazino}-2′-hydroxy-biphenyl-3-carboxylic acid (Compound 206);
3′-{N′-[5,7-Difluoro-2-oxo-1-(4-propyl-phenyl)-1,2-dihydro-indol-3-ylidene]-hydrazino}-2′-hydroxy-biphenyl-3-carboxylic acid (Compound 207);
3′-{N′-[5,7-Difluoro-1-(4-isopropyl-phenyl)-2-oxo-1,2-dihydro-indol-3-ylidene]-hydrazino}-2′-hydroxy-biphenyl-3-carboxylic acid (Compound 208);
3′-{N′-[1-(3,4-Dimethyl-phenyl)-2-oxo-6-trifluoromethyl-1,2-dihydro-indol-3-ylidene]-hydrazino}-2′-hydroxy-biphenyl-3-carboxylic acid (Compound 209);
3′-{N′-[1-(3,4-Dimethyl-phenyl)-6-ethyl-2-oxo-1,2-dihydro-indol-3-ylidene]-hydrazino}-2′-hydroxy-biphenyl-3-carboxylic acid (Compound 210);
3′-{N′-[1-(3,4-Dimethyl-phenyl)-6-methoxy-2-oxo-1,2-dihydro-indol-3-ylidene]-hydrazino}-2′-hydroxy-biphenyl-3-carboxylic acid (Compound 211);
3′-{N′-[5-Chloro-1-(3,4-dimethyl-phenyl)-2-oxo-6-trifluoromethyl-1,2-dihydro-indol-3-ylidene]-hydrazino}-2′-hydroxy-biphenyl-3-carboxylic acid (Compound 212);
3′-{N′-[1-(3,4-Dimethyl-phenyl)-6,7-dimethyl-2-oxo-1,2-dihydro-indol-3-ylidene]-hydrazino}-2′-hydroxy-biphenyl-3-carboxylic acid (Compound 213);
2-(3′-{N′-[1-(3,4-Dimethyl-phenyl)-2-oxo-6-trifluoromethyl-1,2-dihydro-indol-3-ylidene]-hydrazino}-2′-hydroxy-biphenyl-4-yl)-2-methyl-propionic acid (Compound 214);
(−)-2-(3′-{N′-[1-(3,4-Dimethyl-phenyl)-2-oxo-6-trifluoromethyl-1,2-dihydro-indol-3-ylidene]-hydrazino}-2′-hydroxy-biphenyl-4-yl)-propionic acid (Compound 215) and (+)-2-(3′-{N′-[1-(3,4-Dimethyl-phenyl)-2-oxo-6-trifluoromethyl-1,2-dihydro-indol-3-ylidene]-hydrazino}-2′-hydroxy-biphenyl-4-yl)-propionic acid (Compound 215a);
(±)-(3′-{N′-[1-(3,4-Dimethyl-phenyl)-2-oxo-6-trifluoromethyl-1,2-dihydro-indol-3-ylidene]-hydrazino}-2′-hydroxy-5′-methyl-biphenyl-4-yl)-propionic acid (Compound 216);
(±)-2-(3′-{N′-[1-(3,4-Dimethyl-phenyl)-2-oxo-6-trifluoromethyl-1,2-dihydro-indol-3-ylidene]-hydrazino}-5′-fluoro-2′-hydroxy-biphenyl-4-yl)-propionic acid (Compound 217);
5-(4-{N′-[1-(3,4-Dimethyl-phenyl)-5,7-difluoro-2-oxo-1,2-dihydro-indol-3-ylidene]-hydrazino}-3-hydroxy-benzylidene)-thiazolidine-2,4-dione (Compound 218);
5-(4-{N′-[1-(4-Ethyl-phenyl)-5,7-difluoro-2-oxo-1,2-dihydro-indol-3-ylidene]-hydrazino}-3-hydroxy-benzylidene)-thiazolidine-2,4-dione (Compound 219);
5-(4-{N′-[5,7-Difluoro-2-oxo-1-(4-propyl-phenyl)-1,2-dihydro-indol-3-ylidene]-hydrazino}-3-hydroxy-benzylidene)-thiazolidine-2,4-dione (Compound 220);
5-(3-Hydroxy-4-{N′-[1-(4-isopropyl-phenyl)-2-oxo-6-trifluoromethyl-1,2-dihydro-indol-3-ylidene]-hydrazino}-benzylidene)-thiazolidine-2,4-dione (Compound 221);
5-(3-Hydroxy-4-{N′-[1-(4-isopropyl-phenyl)-2-oxo-5,7-difluoro-1,2-dihydro-indol-3-ylidene]-hydrazino}-benzylidene)-thiazolidine-2,4-dione (Compound 222);
3′-{N′-[1-(3,4-Dimethyl-phenyl)-2-oxo-6-trifluoromethyl-1,2-dihydro-indol-3-ylidene]-hydrazino}-5′-fluoro-2′-hydroxy-biphenyl-3-carboxylic acid (Compound 223);
5′-Chloro-3′-{N′-[1-(3,4-dimethyl-phenyl)-2-oxo-6-trifluoromethyl-1,2-dihydro-indol-3-ylidene]-hydrazino}-2′-hydroxy-biphenyl-3-carboxylic acid (Compound 224);
3′-{N′-[1-(3,4-Dimethyl-phenyl)-2-oxo-6-trifluoromethyl-1,2-dihydro-indol-3-ylidene]-hydrazino}-2′-hydroxy-5′-methyl-biphenyl-3-carboxylic acid (Compound 225);
2′-Hydroxy-3′-{N′-[2-oxo-6-trifluoromethyl-1-(4-trifluoromethyl-phenyl)-1,2-dihydro-indol-3-ylidene]-hydrazino}-biphenyl-3-carboxylic acid (Compound 226);
3′-{N′-[1-(4-Ethyl-3-methyl-phenyl)-2-oxo-6-trifluoromethyl-1,2-dihydro-indol-3-ylidene]-hydrazino}-2′-hydroxy-biphenyl-3-carboxylic acid (Compound 227);
3′-{N′-[1-(4-Chloro-3-trifluoromethyl-phenyl)-2-oxo-6-trifluoromethyl-1,2-dihydro-indol-3-ylidene]-hydrazino}-2′-hydroxy-biphenyl-3-carboxylic acid (Compound 228);
3′-{N′-[1-(3,5-Dimethyl-phenyl)-2-oxo-6-trifluoromethyl-1,2-dihydro-indol-3-ylidene]-hydrazino}-5′-fluoro-2′-hydroxy-biphenyl-3-carboxylic acid (Compound 229);
3′-{N′-[1-(3,4-Dimethyl-phenyl)-2-oxo-6-trifluoromethyl-1,2-dihydro-indol-3-ylidene]-hydrazino}-4,5′-difluoro-2′-hydroxy-biphenyl-3-carboxylic acid (Compound 230);
3′-{N′-[1-(3,5-Dimethyl-phenyl)-2-oxo-6-trifluoromethyl-1,2-dihydro-indol-3-ylidene]-hydrazino}-4,5′-difluoro-2′-hydroxy-biphenyl-3-carboxylic acid (Compound 231);
4,5′-Difluoro-2′-hydroxy-3′-{N′-[2-oxo-6-trifluoromethyl-1-(4-trifluoromethyl-phenyl)-1,2-dihydro-indol-3-ylidene]-hydrazino}-biphenyl-3-carboxylic acid (Compound 232);
3′-{N′-[1-(4-Fluoro-3,5-dimethyl-phenyl)-2-oxo-6-trifluoromethyl-1,2-dihydro-indol-3-ylidene]-hydrazino}-2′-hydroxy-biphenyl-3-carboxylic acid (Compound 233);
2′-Hydroxy-3′-{N′-[1-(4-methoxy-phenyl)-2-oxo-6-trifluoromethyl-1,2-dihydro-indol-3-ylidene]-hydrazino}-biphenyl-3-carboxylic acid (Compound 234);
3′-{N′-[1-(4-Fluoro-phenyl)-2-oxo-6-trifluoromethyl-1,2-dihydro-indol-3-ylidene]-hydrazino}-2′-hydroxy-biphenyl-3-carboxylic acid (Compound 235);
3′-{N′-[1-(3,5-Dimethoxy-phenyl)-2-oxo-6-trifluoromethyl-1,2-dihydro-indol-3-ylidene]-hydrazino}-2′-hydroxy-biphenyl-3-carboxylic acid (Compound 236);
3′-{N′-[1-(3,4-Dimethoxy-phenyl)-2-oxo-6-trifluoromethyl-1,2-dihydro-indol-3-ylidene]-hydrazino}-2′-hydroxy-biphenyl-3-carboxylic acid (Compound 237);
3′-{N′-[1-(3,5-Difluoro-phenyl)-2-oxo-6-trifluoromethyl-1,2-dihydro-indol-3-ylidene]-hydrazino}-2′-hydroxy-biphenyl-3-carboxylic acid (Compound 238);
5′-Fluoro-3′-{N′-[1-(4-fluoro-3,5-dimethyl-phenyl)-2-oxo-6-trifluoromethyl-1,2-dihydro-indol-3-ylidene]-hydrazino}-2′-hydroxy-biphenyl-3-carboxylic acid (Compound 239);
4,5′-Difluoro-3′-{N′-[1-(4-fluoro-3,5-dimethyl-phenyl)-2-oxo-6-trifluoromethyl-1,2-dihydro-indol-3-ylidene]-hydrazino}-2′-hydroxy-biphenyl-3-carboxylic acid (Compound 240);
2′-Hydroxy-3′-{N′-[1-(4-methoxy-3,5-dimethyl-phenyl)-2-oxo-6-trifluoromethyl-1,2-dihydro-indol-3-ylidene]-hydrazino}-biphenyl-3-carboxylic acid (Compound 241);
2′-Hydroxy-3′-{N′-[1-(4-hydroxy-3,5-dimethyl-phenyl)-2-oxo-6-trifluoromethyl-1,2-dihydro-indol-3-ylidene]-hydrazino}-biphenyl-3-carboxylic acid (Compound 242);
3′-{N′-[1-(4-Cyclohexyl-phenyl)-2-oxo-1,2-dihydro-indol-3-ylidene]-hydrazino}-2′-hydroxy-biphenyl-3-carboxylic acid (Compound 243);
2′-Hydroxy-3′-[N′-(2-oxo-1-pyridin-2-yl-1,2-dihydro-indol-3-ylidene)-hydrazino]-biphenyl-3-carboxylic acid (Compound 244);
2′-Hydroxy-3′-[N′-(2-oxo-1-pyridin-3-yl-1,2-dihydro-indol-3-ylidene)-hydrazino]-biphenyl-3-carboxylic acid (Compound 245);
3′-{N′-[1-(4-Ethyl-phenyl)-2-oxo-6-trifluoromethyl-1,2-dihydro-indol-3-ylidene]-hydrazino}-2′-hydroxy-biphenyl-3-carboxylic acid (Compound 246);
3′-{N′-[1-(4-Ethyl-phenyl)-4-fluoro-2-oxo-6-trifluoromethyl-1,2-dihydro-indol-3-ylidene]-hydrazino}-2′-hydroxy-biphenyl-3-carboxylic acid (Compound 247);
3-[(3′-Carboxy-2-hydroxy-biphenyl-3-yl)-hydrazono]-1-(3,5-dimethyl-phenyl)-2-oxo-2,3-dihydro-1-H-indole-5-carboxylic acid methyl ester (Compound 248);
3′-{N′-[1-(3-Chloro-4-methyl-phenyl)-2-oxo-6-trifluoromethyl-1,2-dihydro-indol-3-ylidene]-hydrazino}-2′-hydroxy-biphenyl-3-carboxylic acid (Compound 249);
5-(4-{N′-[1-(3,5-Dimethyl-phenyl)-2-oxo-6-trifluoromethyl-1,2-dihydro-indol-3-ylidene]-hydrazino}-3-hydroxy-benzylidene)-thiazolidine-2,4-dione (Compound 250);
2′-Hydroxy-3′-(N′-{2-oxo-1-[4-(4,4,4-trifluoro-butyl)-phenyl]-1,2-dihydro-indol-3-ylidene}-hydrazine)-biphenyl-3-carboxylic acid (Compound 251);
3′-{N′-[1-(3,5-Dimethyl-phenyl)-4-fluoro-2-oxo-6-trifluoromethyl-1,2-dihydro-indol-3-ylidene]-hydrazino}-2′-hydroxy-biphenyl-3-carboxylic acid (Compound 252);
3′-{N′-[1-(4-tert-Butyl-phenyl)-2-oxo-6-trifluoromethyl-1,2-dihydro-indol-3-ylidene]-hydrazino}-2′-hydroxy-biphenyl-3-carboxylic acid (Compound 253);
3′-{N′-[1-(3,4-Dimethyl-phenyl)-2-oxo-6-trifluoromethyl-1,2-dihydro-indol-3-ylidene]-hydrazino}-2′-hydroxy-biphenyl-4-carboxylic acid (Compound 254);
3′-{N′-[1-(3,4-Dimethyl-phenyl)-2-oxo-6-bromo-1,2-dihydro-indol-3-ylidene]-hydrazino}-2′-hydroxy-biphenyl-3-carboxylic acid (Compound 255);
3′-{N′-[1-(3,4-Dimethyl-phenyl)-2-oxo-6-trifluoromethyl-1,2-dihydro-indol-3-ylidene]-hydrazino}-3-fluoro-2′-hydroxy-biphenyl-4-carboxylic acid (Compound 256);
3′-{N′-[1-(3,5-Bis-trifluoromethyl-phenyl)-2-oxo-6-trifluoromethyl-1,2-dihydro-indol-3-ylidene]-hydrazino}-2′-hydroxy-biphenyl-3-carboxylic acid (Compound 257);
3′-{N′-[1-(3,4-Dichloro-phenyl)-2-oxo-6-trifluoromethyl-1,2-dihydro-indol-3-ylidene]-hydrazino}-2′-hydroxy-biphenyl-3-carboxylic acid (Compound 258);
3′-{N′-[1-(3,5-Dichloro-phenyl)-2-oxo-6-trifluoromethyl-1,2-dihydro-indol-3-ylidene]-hydrazino}-2′-hydroxy-biphenyl-3-carboxylic acid (Compound 259);
3-(4-{N′-[1-(3,5-Dimethyl-phenyl)-2-oxo-6-trifluoromethyl-1,2-dihydro-indol-3-ylidene]-hydrazino}-3-hydroxy-phenyl)-2-methyl-acrylic acid (Compound 260);
3-(4-{N′-[1-(3,4-Dimethyl-phenyl)-2-oxo-6-trifluoromethyl-1,2-dihydro-indol-3-ylidene]-hydrazino}-3-hydroxy-phenyl)-2-methyl-acrylic acid (Compound 261);
2′-Hydroxy-3′-[N′-(2-oxo-7-phenyl-1,2-dihydro-indol-3-ylidene)-hydrazino]-biphenyl-3-carboxylic acid (Compound 262);
3′-{N′-[1-(3,4-Dimethyl-phenyl)-2-oxo-6-trifluoromethoxy-1,2-dihydro-indol-3-ylidene]-hydrazino}-2′-hydroxy-biphenyl-3-carboxylic acid (Compound 263);
3′-{N′-[1-(3,4-Dimethyl-phenyl)-2-oxo-6-(1,1,2,2-tetrafluoro-ethoxy)-1,2-dihydro-indol-3-ylidene]-hydrazino}-2′-hydroxy-biphenyl-3-carboxylic acid (Compound 264);
3′-{N′-[1-(3,4-Dimethyl-phenyl)-5-methyl-2-oxo-1,2-dihydro-indol-3-ylidene]-hydrazino}-2′-hydroxy-biphenyl-3-carboxylic acid (Compound 265);
3′-{N′-[1-(4-Isopropyl-phenyl)-5-methyl-2-oxo-1,2-dihydro-indol-3-ylidene]-hydrazino}-2′-hydroxy-biphenyl-3-carboxylic acid (Compound 266);
3′-{N′-[1-(3,4-Dimethyl-phenyl)-6-phenyl-2-oxo-1,2-dihydro-indol-3-ylidene]-hydrazino}-2′-hydroxy-biphenyl-3-carboxylic acid (Compound 267);
3′-{N′-[1-(3-Trifluoromethyl-phenyl)-6-trifluoromethyl-2-oxo-1,2-dihydro-indol-3-ylidene]-hydrazino}-2′-hydroxy-biphenyl-3-carboxylic acid (Compound 268);
3′-{N′-[1-(4-Trifluoromethoxy-phenyl)-5-trifluoromethoxy-2-oxo-1,2-dihydro-indol-3-ylidene]-hydrazino}-2′-hydroxy-biphenyl-3-carboxylic acid (Compound 269);
3′-{N′-[1-(3,5-Dimethyl-phenyl)-6-trifluoromethyl-2-oxo-1,2-dihydro-indol-3-ylidene]-hydrazino}-2′-hydroxy-biphenyl-3-carboxylic acid (Compound 270);
3′-{N′-[1-(3-Trifluoromethyl-phenyl)-4,6-dimethyl-2-oxo-1,2-dihydro-indol-3-ylidene]-hydrazino}-2′-hydroxy-biphenyl-3-carboxylic acid (Compound 271);
3′-{N′-[1-(3-Trifluoromethyl-phenyl)-5,6-dimethyl-2-oxo-1,2-dihydro-indol-3-ylidene]-hydrazino}-2′-hydroxy-biphenyl-3-carboxylic acid (Compound 272);
3′-{N′-[1-(3,5-Dimethyl-phenyl)-6-trifluoromethyl-2-oxo-1,2-dihydro-indol-3-ylidene]-hydrazino}-2′-hydroxy-5′-chloro-4-fluoro-biphenyl-3-carboxylic acid (Compound 273);
3′-{N′-[1-(3,5-Dimethyl-phenyl)-6-trifluoromethyl-2-oxo-1,2-dihydro-indol-3-ylidene]-hydrazino}-2′-hydroxy-4-fluoro-biphenyl-3-carboxylic acid (Compound 274);
3′-{N′-[6-Chloro-1-(3,4-dimethyl-phenyl)-2-oxo-1,2-dihydro-indol-3-ylidene]-hydrazino}-2′-hydroxy-biphenyl-3-carboxylic acid (Compound 275);
3′-{N′-[5-Fluoro-2-oxo-1-(4-propyl-phenyl)-1,2-dihydro-indol-3-ylidene]-hydrazino}-2′-hydroxy-biphenyl-3-carboxylic acid (Compound 276);
3′-{N′-[5-Cyano-1-(3,4-dimethyl-phenyl)-2-oxo-1,2-dihydro-indol-3-ylidene]-hydrazino}-2′-hydroxy-biphenyl-3-carboxylic acid (Compound 277);
3′-{N′-[6-Chloro-1-(3,5-dimethyl-phenyl)-2-oxo-1,2-dihydro-indol-3-ylidene]hydrazino}-2′-hydroxy-biphenyl-3-carboxylic acid (Compound 278);
4-Fluoro-3′-{N′-[1-(3-fluoro-4-methyl-phenyl)-2-oxo-6-trifluoromethyl-1,2-dihydro-indol-3-ylidene]-hydrazino}-2′-hydroxy-biphenyl-3-carboxylic acid (Compound 279);
3′-{N′-[1-(4-Chloro-3,5-dimethylphenyl)-2-oxo-6-trifluoromethyl-1,2-dihydroindol-3-ylidene]hydrazino}-2′-hydroxy-biphenyl-3-carboxylic acid (Compound 280);
3′-{N′-[1-(3,5-Dimethylphenyl)-4-fluoro-2-oxo-6-trifluoromethyl-1,2-dihydroindol-3-ylidene]hydrazino}-2′-hydroxybiphenyl-4-fluoro-3-carboxylic acid (Compound 281);
3′-{N′-[1-Benzo[1,3]dioxo-5-yl-2-oxo-6-trifluoromethyl-1,2-dihydroindol-3-ylidene]hydrazino}-2′-hydroxybiphenyl-3-carboxylic acid (Compound 282);
3′-{N′-[1-Benzo[1,3]dioxo-5-yl-2-oxo-6-trifluoromethyl-1,2-dihydroindol-3-ylidene]hydrazino}-2′-hydroxybiphenyl-2-fluoro-3-carboxylic acid (Compound 283);
3′-{N′-[1-(3,5-Dimethylphenyl)-2-oxo-6-trifluoromethyl-1,2-dihydroindol-3-ylidene]hydrazino}-2′-hydroxybiphenyl-2-hydroxy-3-carboxylic acid (Compound 284);
3′-{N′-[1-(3-Methoxycarbonylphenyl)-2-oxo-6-trifluoromethyl-1,2-dihydroindol-3-ylidene]hydrazino}-2′-hydroxybiphenyl-3-carboxylic acid (Compound 285);
3′-{N′-[1-(3-Methoxycarbonylphenyl)-2-oxo-1,2-dihydroindol-3-ylidene]hydrazino}-2′-hydroxybiphenyl-3-carboxylic acid (Compound 286);
3′-{N′-[7-Aza-1-(3,4-dimethylphenyl)-2-oxo-1,2-dihydroindol-3-ylidene]hydrazino}-2′-hydroxybiphenyl-3-carboxylic acid (Compound 287);
3′-{N′-[1-(3,5-Dimethylphenyl)-2-oxo-1,2-dihydroindol-6-trifluoromethyl-3-ylidene]hydrazino}-2′-hydroxybiphenyl-3-(2-methyl-2-propionic acid) (Compound 288);
3′-{N′-[1,3-N,N-Dimethylbarbitur-5-ylidene]hydrazino}-2′-hydroxybiphenyl-3-carboxylic acid (Compound 289);
3′-{N′-[1-N-(4-Trifluoromethylbenzyl)-2,8-dioxo-1,2,7,8-tetrahydroisoquinolin-7-ylidene]hydrazino}-2′-hydroxybiphenyl-3-carboxylic acid (Compound 290);
3′-{N′-[1-N-(4-Methylbenzyl)-2,8-dioxo-1,2,7,8-tetrahydroisoquinolin-7-ylidene]hydrazino}-2′-hydroxybiphenyl-3-carboxylic acid (Compound 291);
3′-{N′-[1-N-Benzyl-2,8-dioxo-1,2,7,8-tetrahydroisoquinolin-7-ylidene]hydrazino}-2′-hydroxybiphenyl-3-carboxylic acid (Compound 292);
3′-{N′-[1-N-(4-Trifluoromethylphenyl)-2,8-dioxo-1,2,7,8-tetrahydroisoquinolin-7-ylidene]hydrazino}-2′-hydroxybiphenyl-3-carboxylic acid (Compound 293);
3′-{N′-[1-N-(3-Trifluoromethylphenyl)-2,8-dioxo-1,2,7,8-tetrahydroisoquinolin-7-ylidene]hydrazino}-2′-hydroxybiphenyl-3-carboxylic acid (Compound 294);
3′-{N′-[1-N-(3,5-Dimethylphenyl)-2,8-dioxo-1,2,7,8-tetrahydroisoquinolin-7-ylidene]hydrazino}-2′-hydroxybiphenyl-3-carboxylic acid (Compound 295);
3′-{N′-[1-N-Phenyl-2,8-dioxo-1,2,7,8-tetrahydroisoquinolin-7-ylidene]hydrazino}-2′-hydroxybiphenyl-3-carboxylic acid (Compound 296);
3′-{N′-[1-N-(3,4-Dimethylphenyl)-2,8-dioxo-1,2,7,8-tetrahydroisoquinolin-7-ylidene]hydrazino}-2′-hydroxybiphenyl-3-carboxylic acid (Compound 297);
3′-{N′-[1-N-(3,4-Dimethylphenyl)-2-oxo-6-trifluoromethyl-1,2-dihydroindol-3-ylidene]hydrazino}-2′-fluorobiphenyl-3-carboxylic acid (Compound 298);
3-(3-{N′-[1-N-(3,4-Dimethylphenyl)-2-oxo-6-trifluoromethyl-1,2-dihydroindol-3-ylidene]hydrazino}-2-hydroxyphenyl)-2(Z)-propenoic acid (Compound 299);
3-(3-{N′-[1-N-(3,4-Dimethylphenyl)-2-oxo-4-fluoro-6-trifluoromethyl-1,2-dihydroindol-3-ylidene]hydrazino}-2-hydroxyphenyl)-2(Z)-propenoic acid (Compound 300);
5-(3-{N′-[1-(3,4-Dimethylphenyl)-2-oxo-4-fluoro-6-trifluoromethyl-1,2-dihydroindol-3-ylidene]hydrazino}-2-hydroxybenzylidene)thiazolidine-2,4-dione (Compound 301);
2-Chloro-3-(4-{N′-[1-(3,4-dimethylphenyl)-2-oxo-6-trifluoromethyl-1,2-dihydroindol-3-ylidene]hydrazino}-3-hydroxyphenyl)-2-propenoic acid (Compound 302);
2-Ethyl-3-(4-{N′-[1-(3,4-dimethylphenyl)-2-oxo-6-trifluoromethyl-1,2-dihydroindol-3-ylidene]hydrazino}-3-hydroxyphenyl)-2-propenoic acid (Compound 303);
1-N-Methyl-5-(4-{N′-[1-(3,5-dimethylphenyl)-2-oxo-6-trifluoromethyl-1,2-dihydroindol-3-ylidene]hydrazino}-3-hydroxybenzylidene)-1,3-diazolidine-2,4-dione (Compound 304);
5-(4-{N′-[1-(3,5-Dimethylphenyl)-2-oxo-6-trifluoromethyl-1,2-dihydroindol-3-ylidene]hydrazino}-3-hydroxybenzylidene)-1,3-diazolidine-2,4-dione (Compound 305);
2-Fluoro-3-(4-{N′-[1-(3,4-dimethylphenyl)-2-oxo-6-trifluoromethyl-1,2-dihydroindol-3-ylidene]hydrazino}-3-hydroxyphenyl)-2-propenoic acid (Compound 306);
(±)-2-Methoxy-3-(4-{N′-[1-(3,5-dimethylphenyl)-2-oxo-6-trifluoromethyl-1,2-dihydroindol-3-ylidene]hydrazino}-3-hydroxyphenyl)propanoic acid (Compound 307);
4-(3-{N′-[1-(3,4-dimethylphenyl)-2-oxo-6-trifluoromethyl-1,2-dihydroindol-3-ylidene]hydrazino}-2-hydroxyphenyl)butanoic acid (Compound 308);
3-(2-{N′-[1-(3,5-dimethylphenyl)-2-oxo-6-trifluoromethyl-1,2-dihydroindol-3-ylidene]hydrazino}-3-hydroxyphenoxy)propanoic acid (Compound 309);
4-(4-{N′-[1-(3,4-dimethylphenyl)-2-oxo-6-trifluoromethyl-1,2-dihydroindol-3-ylidene]hydrazino}-3-hydroxyphenyl)butanoic acid (Compound 310); and
   a pharmaceutically acceptable salt ester, amide or prodrug of any of those compounds. Structures and NMR data for those compounds may be found in U.S. Ser. No. 11/256,572.

Certain compounds of the present inventions may exist as stereoisomers including optical isomers. The present disclosure is intended to include all stereoisomers and both the racemic mixtures of such stereoisomers as well as the individual enantiomers that may be separated according to methods that are known in the art or that may be excluded by synthesis schemes known in the art designed to yield predominantly one enantomer relative to another.

### Certain Synthesis Methods

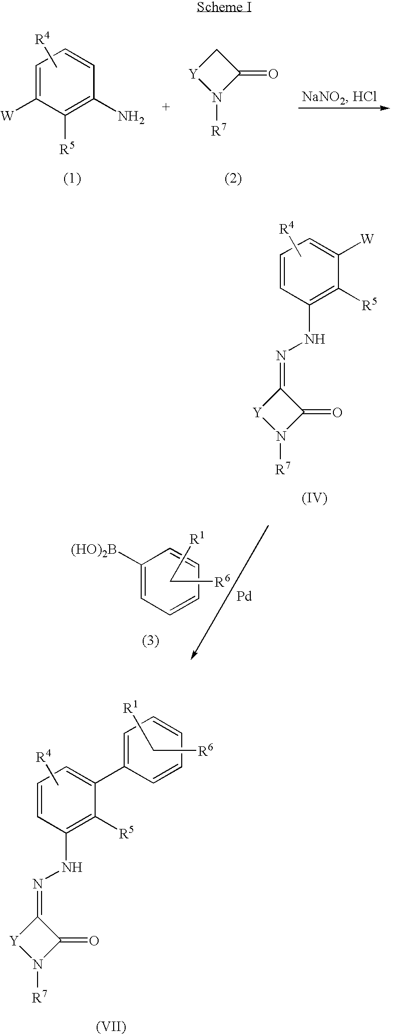

In certain embodiments of Scheme I, W is a halogen. In certain such embodiments, the process of Scheme I begins by treatment of a halo substituted aminophenyl (1), for example, 6-bromo-4-aminophenol, with sodium nitrite in HCl followed by treatment with an oxo nitrogen containing heterocycle such as an oxindole (2) (e.g., 6-(trifluoromethyl)-1-(3,5-dimethylphenyl) oxindole). The resulting compound (IV) can then be treated with a carboxyphenyl boronic acid derivative (3) under a metal catalyzed condition, for example, 3-carboxyphenylboronic acid, to afford the final product (VII).

In certain embodiments, W is a metal, for example, a boronic acid or trialkylstannane. When W is a metal, compound (1) can be treated with an oxidizing agent such as sodium nitrite in HCl followed by treatment with an oxo nitrogen containing heterocycle (2) such as an oxindole. The resulting compound (IV) can then be treated with a 3-halobenzoic acid derivative (3) under a metal catalyzed condition, for example, 3-bromobenzoic acid, to afford the final product (VII).

In certain embodiments of Scheme 1, R⁵ is a hydroxy protected with a protection group such as methyl, acetate, or CH₂OCH₃. The protection group can be optionally introduced on compounds of structure (1). Alternatively, the protection group may be introduced on the compound of structure (IV) prior to conversion to a protected version of structure (VII), after which the unprotected version of structure (VII) may be formed by deprotection of the hydroxy. Protection of R⁵ when it is hydroxy may be accomplished by methods known in the art (e.g., by reaction with CH₃OCH₂Cl).

One of skill in the art will recognize that analogous synthesis schemes may be used to synthesize similar compounds. In certain embodiments, the invention provides a salt corresponding to any of the compounds provided herein.

In certain embodiments, the invention provides a salt corresponding to a selective TPO modulator. In certain embodiments, the invention provides a salt corresponding to a selective TPO receptor binding agent. In certain embodiments, a salt is obtained by reacting a compound with an acid, such as hydrochloric acid, hydrobromic acid, sulfuric acid, nitric acid, phosphoric acid, methanesulfonic acid, ethanesulfonic acid, p-toluenesulfonic acid, salicylic acid, and the like. In certain embodiments, a salt is obtained by reacting a compound with a base to form a salt such as an ammonium salt, an alkali metal salt, such as a sodium or a potassium salt, an alkaline earth metal salt, such as a calcium or a magnesium salt, a salt of organic bases such as choline, dicyclohexylamine, N-methyl-D-glucamine, tris(hydroxymethyl)methylamine, 4-(2-hydroxyethyl)-morpholine, 1-(2-hydroxyethyl)-pyrrolidine, ethanolamine and salts with amino acids such as arginine, lysine, and the like. In certain embodiments, a salt is obtained by reacting a free acid form of a selective TPO modulator or selective TPO binding agent with multiple molar equivalents of a base, such as bis-sodium, bis-ethanolamine, and the like.

In certain embodiments, a salt corresponding to a compound of the present invention is selected from acetate, ammonium, benzenesulfonate, benzoate, bicarbonate, bisulfate, bitartrate, borate, bromide, calcium edetate, camsylate, carbonate, chloride, cholinate, clavulanate, citrate, dihydrochloride, diphosphate, edetate, edisylate, estolate, esylate, fumarate, gluceptate, gluconate, glutamate, glycollylarsanilate, hexylresorcinate, hydrabanine, hydrobromi de, hydrochloride, hydroxynaphthoate, iodide, isethionate, lactate, lactobionate, laurate, magnesium, malate, maleate, mandelate, mucate, napsylate, nitrate, N-methylglucamine, oxalate, pamoate (embonate), palmitate, pantothenate, phosphate, polygalacturonate, potassium, salicylate, sodium, stearate, subaceatate, succinate, sulfate, tannate, tartrate, teoclate, tosylate, triethiodide, tromethamine, trimethylammonium, and valerate salts.

### Certain Intermediates

Certain embodiments include intermediates obtained during the above-described synthetic processes. In one embodiment, an intermediate having the following structure is provided:

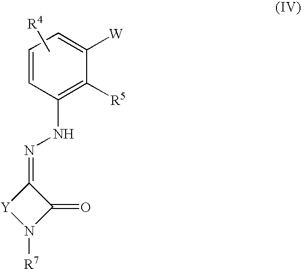

where W is selected from a halogen, B(OH)₂, B(OR^{A})₂, Sn(R^{B})₃ where each R^{A} is selected from an optionally substituted C₁-C₆ aliphatic; or the two OR^{A} groups together form an optionally substituted ring; and R^{B} is selected from an optionally substituted C₁-C₆ aliphatic, or an optionally substituted phenyl, or an optionally substituted heteroaryl:

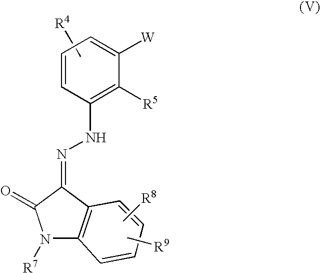

where R⁸ and R⁹ are as defined above. In one such embodiment, the invention provides a compound having the structure:

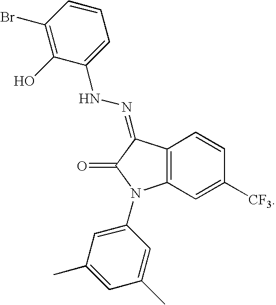

In certain embodiments, such compounds are useful as intermediates for making TPO modulators. In certain embodiments, such compounds may, themselves, be useful as TPO modulators, TPO mimics, and/or TPO binding agents.

In certain embodiments, one or more carbon atoms of a compound of the present invention are replaced with silicon. See e.g., WO 03/037905A1; Tacke and Zilch, Endeavour, New Series, 10, 191-197 (1986); and Bains and Tacke, Curr. Opin. Drug Discov Devel. July:6(4):526-43 (2003). In certain embodiments, compounds of the present invention comprising one or more silicon atoms possess certain desired properties, including, but not limited to, greater stability and/or longer half-life in a patient, when compared to the same compound in which none of the carbon atoms have been replaced with a silicon atom.

### Certain Assays

In certain embodiments, compounds of the present invention and compounds made using the methods of the present invention may be used in a any of a variety of assays. For example, compounds of the present invention may be tested for potency as selective TPO modulators in a luciferase assay, such as those described in Lamb, et al., Nucleic Acids Research, 23: 3283-3289 (1995) and/or Seidel et al, Proc. Nat. Acad. Sci. USA; 92: 3041-3045 (1995).

Certain compounds of the present invention may be used in in vitro proliferation and/or differentiation assays, such as those described by Bartley et al., Cell, 77: 1117-1124 (1994) and/or Cwirla, et al., Science, 276: 1696-1699 (1997).

### EXAMPLES

The following examples, including experiments and results achieved, are provided for illustrative purposes only and are not to be construed as limiting the present invention.

### Example 1

### Synthesis of 6-Trifluoromethyloxindole

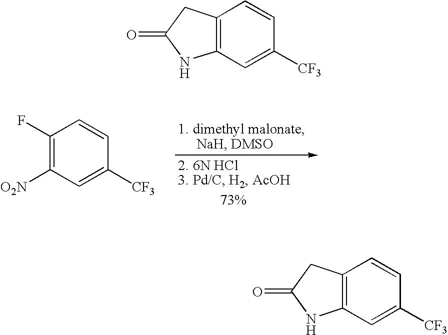

To prepare 6-trifluoromethyloxindole, first a 2 L flask with a stir bar was charged with 45.8 ml (400 mmol) of dimethylmalonate and 500 ml of anhydrous DMSO. Next, 15.6 grams (391 mmol) of NaH was added in portions over 10 minutes to the vigorously stirring solution under an atmosphere of N2. That solution was heated to 100° C. and stirred for 1 hour and then allowed to cool to ambient temperature. Next, 26 ml (186 mmol) of 4-fluoro-3-nitrobenzotrifluoride (CAS#367-86-2) was added using a syringe in one portion, which resulted in the previously colorless solution becoming dark brown/red. That colored solution was again heated to 100° C., stirred for 1 hour and allowed to cool to ambient temperature. The solution was then poured into 1.3 L of saturated NH₄Cl solution. The resulting mixture was Extracted with ethyl acetate followed by drying (using MgSO₄) and concentration in vacuo, resulting in a red/orange oil that crystallized on standing overnight. Some of the excess dimethylmalonate was removed by decanting from the crystallized solid product. The crystallized solid product was then pulverized using a mortar and pestle, suspended in hexanes and filtered to remove the remaining dimethylmalonate.

The resulting 2-(2-Nitro-4-trifluoromethyl-phenyl)-malonic acid dimethyl ester (56.1 g) was suspended in 200 ml of 6N HCl and stirred at reflux overnight. That solution was cooled, diluted with 500 ml of water, and filtered. The filtered solids were pulverized using mortar and pestil and suspended in water and filtered again, washing with copious water to remove traces of HCl. After drying in vacuo, the resuting solid (2-Nitro-4-trifluoromethyl-phenyl)-acetic acid was dissolved in 200 ml of AcOH to which was then added 5.4 grams of Palladium (10%) on Carbon. The resulting suspension was placed under one atmosphere of hydrogen (60 psi, Parr apparatus) for 4 hours. The suspenson was filtered through celite, washed with MeOH and CH₂Cl₂, and concentrated in vacuo. Recrystallization from ethyl acetate/hexanes gave 27.19 g from the first crop, and 1.1 g from a second crop for a total yield of 28.29 g (76%, 3 steps) of 6-trifluoromethyloxindole (CAS#1735-89-3) as white prisms.

### Example 2

### Synthesis of 1-(3,5-Dimethyl-phenyl)-6-trifluoromethyl-1,3-dihydro-indol-2-one

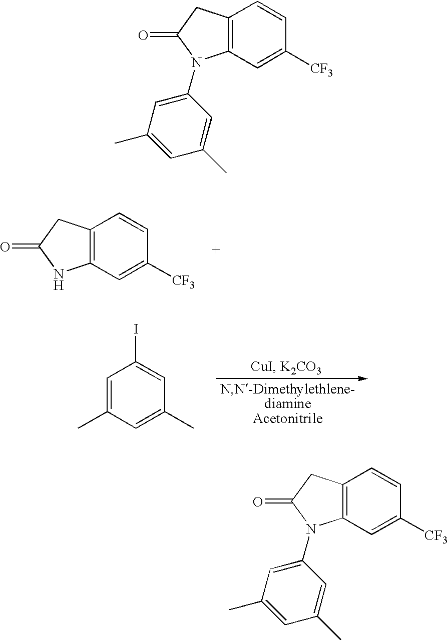

To prepare 1-(3,5-Dimethyl-phenyl)-6-trifluoromethyl-1,3-dihydro-indol-2-one, an oven-dried 3-neck, 5 L round bottomed flask fitted with a reflux condenser and an overhead stirrer was charged with nitrogen. The flask, while under an atmosphere of nitrogen, was then charged with 6-trifluoromethyloxindole (from Example 1), acetonitrile, 5-iodo-m-xylene (CAS#22445-41-6), copper iodide, diamine and potassium carbonate, in that order. The reflux condenser and the unfitted neck of the flask were fitted with rubber septa and the entire system was carefully evacuated. The evacuation was monitored so as not to allow the solution to bump up the neck of the condenser. After approximately 10-20 seconds under vacuum, the system was then back-filled with nitrogen. This process of evacuation and back-filling with nitrogen was repeated twice more. The solution was then heated to a gentle reflux and monitored closely by thin layer chromatography. After four hours, the solution was removed from the heating mantle and was allowed to cool to room temperature. Then, 500 ml of 1 M HCl was added and the resulting solution was diluted with 800 ml of ethyl acetate.

That diluted solution was then poured into a 4 L separatory funnel. Once the layers separated, the aqueous layers were removed and then the organic layer was extracted twice with ethyl acetate. The extracted organic layers were combined and then concentrated by about 80% and allowed to stand overnight at 0° C. The solution was then filtered on a Buchner filter to obtain the solid precipitate. That solid precitpitate was washed with 200 ml of 10% ethyl acetate/hexanes and then transferred to a beaker and suspended in 200 mL of 10% ethyl acetate/hexanes and filtered again on a Buchner filter to give the final product as a beige solid (48 g, one crop, 80%). Pure by HPLC and ¹H NMR; R_{f} (TLC, 20% EtOAc/Hexanes): 0.46.

### Example 3

### Synthesis of 3-[(3-Bromo-2-hydroxy-phenyl)-hydrazono]-1-(3,5-dimethyl-phenyl)-6-trifluoromethyl-1,3-dihydro-indol-2-one

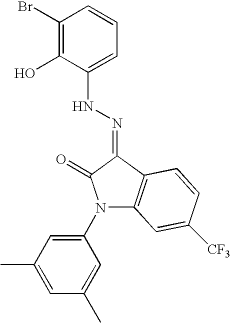

To prepare 3-[(3-Bromo-2-hydroxy-phenyl)-hydrazono]-1-(3,5-dimethyl-phenyl)-6-trifluoromethyl-1,3-dihydro-indol-2-one, first a 25 mL round bottom flask with a stir bar was charged with 230 mg (1.2 mmol) of 2-amino-6-bromophenol (CAS#28165-50-6), 5 ml of ethanol, and 2.4 ml of 1M aqueous hydrochloric acid. That solution was stirred while 1 ml of a solution containing 99 mg (1.4 mmol) of sodium nitrite in water was added slowly. After 10 minutes, 374 mg (1.2 mmol) of 1-(3,5-dimethyl-phenyl)-6-trifluoromethyl-1,3-dihydro-indol-2-one (from Example 2) in ethanol/tetrahydrofuran (5 ml/3 ml) was added in one portion to the stirring solution. Excess potassium carbonate was added until the pH of the mixture was approximately 10. The mixture was then stirred for 30 minutes, and was then poured into ice cold dilute hydrochloric acid. The product was extracted with ethyl acetate, dried over magnesium sulfate, filtered, and concentrated to give the desired product (486 mg, 96%), which required no additional purification. NMR was performed and the following data were obtained:

¹H NMR (500 MHz, acetone-d₆) δ 13.24 (s, NH), 8.80 (s, OH), 7.93 (d, J=8.1 Hz, 1H), 7.80 (dd, J=8.2, 1.3 Hz, 1H), 7.52 (dd, J=7.8, 0.7 Hz, 1H), 7.28 (dd, J=8.1, 1.2 Hz, 1H), 7.19 (dd, J=8.2, 0.6 Hz, 3H), 7.11 (d, J=0.7 Hz, 1H), 6.99 (t, J=8.1 Hz, 1H), 2.41 (d, J=0.7 Hz, 6H).

### Example 4

### Synthesis of 3′-{N′-[1-(3,5-Dimethyl-phenyl)-2-oxo-6-trifluoromethyl-1,2-dihydro-indol-3-ylidene]-hydrazino}-2′-hydroxybiphenyl-3-carboxylic acid

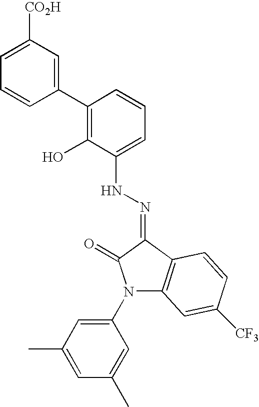

To prepare 3′-{N′-[1-(3,5-Dimethyl-phenyl)-2-oxo-6-trifluoromethyl-1,2-dihydro-indol-3-ylidene]-hydrazino}-2′-hydroxybiphenyl-3-carboxylic acid, first a 35 mL sealable screw-cap tube with a stir bar was charged with 120 mg (0.24 mmol) of 3-[(3-Bromo-2-hydroxy-phenyl)-hydrazono]-1-(3,5-dimethyl-phenyl)-6-trifluoromethyl-1,3-dihydro-indol-2-one (from Example 3), 8 mg (0.0306 mmol, 15 mol %) of palladium acetate, 34 mg (0.072 mmol) of 2-dicyclohexylphosphino-2′,4′,6′-triisopropylbiphenyl (Strem Chemicals, Inc. Newburyport, Mass.); 118 mg (0.71 mmol) of 3-carboxylphenylboronic acid (CAS#14047-29-1), and 41 mg (0.71 mmol) of potassium fluoride. The charged tube was evacuated and back-filled with nitrogen three times; then 2 ml of dioxane was added. The tube was sealed and the mixture was heated to 130° C. for 18 hours. The mixture was then cooled, diluted with 10 ml of diethyl ether, washed with 5 ml of 1 M aqueous hydrochloric acid, and then dried over magnesium sulfate, filtered, evaporated on to silica gel and purified by flash chromatography (gradient from 10% ethyl acetate/90% hexanes to 40% ethyl acetate/60% hexanes, entrained with 1% acetic acid) to give the desired product in 30% yield (60% recovered starting material). NMR was performed and the following data were obtained:

¹H NMR (500 MHz, DMSO-d₆) [symbol] 13.24 (s, 1H), 13.05 (s, 1H), 9.43 (s, 1H), 8.12 (t, J=1.7 Hz, 1H), 7.95 (ddd, J=7.7, 1.7, 1.2 Hz, 1H), 7.93 (d, J=7.8 Hz, 1H), 7.80 (ddd, J=7.7, 1.7, 1.2 Hz, 1H), 7.78 (dd, J=7.7, 1.7 Hz, 1H), 7.61 (t, J=7.7 Hz, 1H), 7.54 (dq, J=7.8, 0.8 Hz, 1H), 7.18-7.16 (m, 3H), 7.14 (t, J=7.7 Hz, 1H), 7.07 (dd, J=7.7, 1.7 Hz, 1H), 6.98 (m, 1H), 2.37 (s, 3H), 2.37 (s, 3H); ¹³C NMR (100 MHz, DMSO-d₆) [symbol] 167.4, 161.3, 141.8, 141.1, 139.7, 138.4, 133.9, 133.6, 133.3, 131.4, 130.8, 130.6, 130.4, 129.3, 128.6, 128.0 (J=31.8 Hz), 125.8, 124.9, 124.8, 123.6 (J=272.3 Hz), 122.4, 120.1 (J=3.3 Hz), 119.4, 113.5, 106.0 (J=3.8 Hz), 21.3.

### Example 5

### Synthesis of 3-[(3-Bromo-2-hydroxy-phenyl)-hydrazono]-1-(4-propyl-phenyl)-1,3-dihydro-indol-2-one

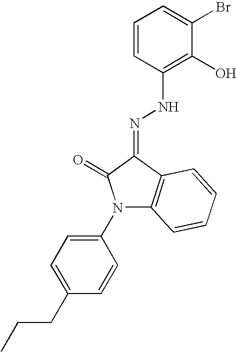

To prepare 3-[(3-Bromo-2-hydroxy-phenyl)-hydrazono]-1-(4-propyl-phenyl)-1,3-dihydro-indol-2-one, approximately 120 mg of 2-amino-6-bromophenol (CAS#28165-50-6) is added to a 25 mL round bottom flask containing a stir bar along with 5 ml of ethanol and 2.5 ml of 1M aqueous hydrochloric acid. The solution is stirred while 1 ml of a solution containing 100 mg of sodium nitrite in water is added slowly. After 10 minutes, 302 mg of 1-(4-propyl-phenyl)-1,3-dihydro-indol-2-one in ethanol/tetrahydrofuran is added in one portion to the stirring solution. Excess sodium carbonate is added until the pH of the mixture is approximately 10. The mixture is then stirred for 30 minutes and poured into ice cold dilute hydrochloric acid. The product is extracted with ethyl acetate, dried over magnesium sulfate, filtered, and concentrated to give the desired product.

### Example 6

### Synthesis of 2′-Hydroxy-3′-{N′-[2-oxo-1-(4-propyl-phenyl)-1,2-dihydro-indol-3-ylidene]-hydrazino}-biphenyl-4-carboxylic acid

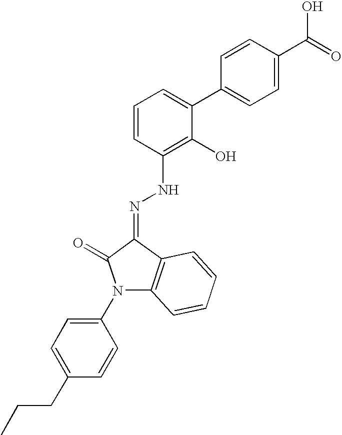

To prepare 2′-Hydroxy-3′-{N′-[2-oxo-1-(4-propyl-phenyl)-1,2-dihydro-indol-3-ylidene]-hydrazino}-biphenyl-4-carboxylic acid, approximately 112 mg of 3-[(3-Bromo-2-hydroxy-phenyl)-hydrazono]-1-(4-propyl-phenyl)-1,3-dihydro-indol-2-one (from Example 5) is added to a 35 mL sealable screw-cap tube with a stir bar along with 8 mg (0.0306 mmol, 15 mol %) of palladium acetate, 33 mg of 2-dicyclohexylphosphino-2′,4′,6′-triisopropylbiphenyl (Strem Chemicals, Inc. Newburyport, Mass.), 114 mg of 4-carboxylphenylboronic acid, and 38 mg of potassium fluoride. The charged tube is evacuated and back-filled with nitrogen three times and then 2 ml of dioxane is added. The tube is sealed and the mixture heated to 130° C. for 18 hours. The mixture is then cooled, diluted with 10 ml of diethyl ether, washed with 5 ml of 1 M aqueous hydrochloric acid, and then dried over magnesium sulfate, filtered, evaporated on to silica gel and purified by flash chromatography (gradient from 10% ethyl acetate/90% hexanes to 40% ethyl acetate/60% hexanes, entrained with 1% acetic acid) to give the desired product.

Although the invention has been described with reference to embodiments and examples, it should be understood that numerous and various modifications can be made without departing from the spirit of the invention. Accordingly, the invention is limited only by the following claims.

What is claimed is:

1. A compound having the structure: 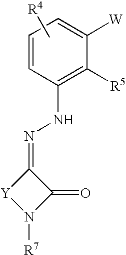
wherein:
R⁴ is selected from hydrogen, F, Cl, Br, C₁-C₄ aliphatic, C₁-C₄ haloaliphatic, C₁-C₄ heteroaliphatic, and a ring;
R⁵ is selected from hydrogen, OR¹⁰, SR¹⁰, NHR¹¹, and CO₂H;
R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R¹⁰ is selected from hydrogen, a protecting group, an optionally substituted C₁-C₄ aliphatic, an optionally substituted C₁-C₄ haloaliphatic, an optionally substituted C₁-C₄ heteroaliphatic, and an optionally substituted ring;
R¹¹ is selected from hydrogen, SO₂R¹⁵, C₁-C₄ aliphatic, C₁-C₄ haloaliphatic, C₁-C₄ heteroaliphatic, and a ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
Y is a 1-4 atom spacer comprising one or more groups selected from an optionally substituted C₁-C₆ aliphatic, an optionally substituted C₁-C₆ heteroaliphatic, an optionally substituted phenyl, an optionally substituted heteroaryl, an optionally substituted C₃-C₅ heterocycle, and an optionally substituted alicyclic;
W is selected from a halogen, B(OH)₂, B(OR^{A})₂, Sn(R^{B})₃ where each R^{A} is selected from an optionally substituted C₁-C₆ aliphatic; or the two OR^{A} groups together form an optionally substituted ring; and R^{B} is selected from an optionally substituted C₁-C₆ aliphatic, or an optionally substituted phenyl, or an optionally substituted heteroaryl; and
m is 0, 1, or 2.

2. The compound of claim 1, wherein Y is a 1-4 atom spacer comprising at least one of:
an optionally substituted C₁-C₆ alkyl, an optionally substituted C₁-C₆ heteroalkyl,
an optionally substituted C₂-C₆ alkenyl, and an optionally substituted C₂-C₆ heteroalkenyl; and
at least one of:
an optionally substituted phenyl an optionally substituted heteroaryl, an optionally substituted C₃-C₅ heterocycle, an optionally substituted cycloalkyl; and an optionally substituted cycloalkenyl.

3. The compound of claim 1, wherein:
Y is selected from: 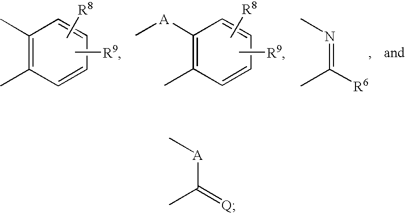
Q is selected from O and S;
A is selected from O, S, NR¹⁰, and CR¹⁰R¹⁰; and
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, heterohaloaliphatic, and a ring.

4. The compound of claim 3, wherein Y is: 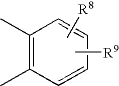
and at least one of R⁸ or R⁹ is C₁-C₄ haloaliphatic.

5. The compound of claim 1, wherein R⁴ is hydrogen.

6. The compound of claim 1, wherein R⁵ is OR¹⁰.

7. The compound of claim 6, wherein R¹⁰ is hydrogen, a C₁-C₄ alkyl, or a C₁-C₄ heteroalkyl.

8. The compound of claim 1, wherein R⁷ is (CH₂)ₘR¹⁴, m is 0, and R¹⁴ is an optionally substituted aryl.

9. The compound of claim 8, wherein R¹⁴ is an optionally substituted phenyl.

10. The compound of claim 1, having the structure: 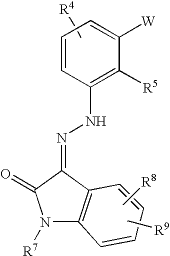
wherein:
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, C₁-C₄ heterohaloaliphatic, and a ring.

11. The compound of claim 1, having the structure:

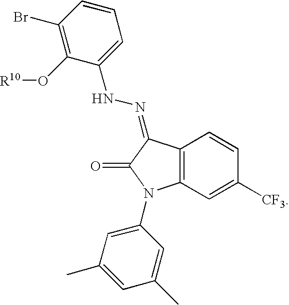

12. The compound of claim 10, wherein R⁴ is hydrogen.

13. The compound of claim 10, wherein R⁵ is hydroxy or alkoxy.

14. The compound of claim 10, wherein R⁷ is an optionally substituted phenyl.

15. A method of obtaining a compound having the structure: 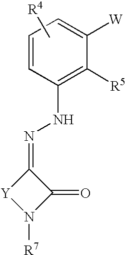
comprising reacting a compound having the structure: 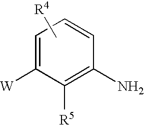
with a nitrite and a compound having the structure: 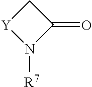
wherein:
R⁴ is selected from hydrogen, F, Cl, Br, C₁-C₄ aliphatic, C₁-C₄ haloaliphatic, C₁-C₄ heteroaliphatic, and a ring;
R⁵ is selected from hydrogen, OR¹⁰, SR¹⁰, NHR¹¹, and CO₂H;
R⁷ is selected from hydrogen, an optionally substituted C₁-C₃ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R¹⁰ is selected from hydrogen, a protecting group, an optionally substituted C₁-C₄ aliphatic, an optionally substituted C₁-C₄ haloaliphatic, an optionally substituted C₁-C₄ heteroaliphatic, and an optionally substituted ring;
R¹¹ is selected from hydrogen, SO₂R¹⁵, C₁-C₄ aliphatic, C₁-C₄ haloaliphatic, heteroaliphatic, and a ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
Y is a 1-4 atom spacer comprising one or more groups selected from an optionally substituted C₁-C₆ aliphatic, an optionally substituted C₁-C₆ heteroaliphatic, an optionally substituted phenyl, an optionally substituted heteroaryl, an optionally substituted C₃-C₅ heterocycle, and an optionally substituted alicyclic;
W is selected from a halogen, B(OH)₂, B(OR^{A})₂, Sn(R^{B})₃ where each R^{A} is selected from an optionally substituted C₁-C₆ aliphatic; or the two OR^{A} groups together form an optionally substituted ring; and R^{B} is selected from an optionally substituted C₁-C₆ aliphatic, or an optionally substituted phenyl, or an optionally substituted heteroaryl; and
m is 0, 1, or 2.

16. The method of claim 15, wherein:
Y is selected from: 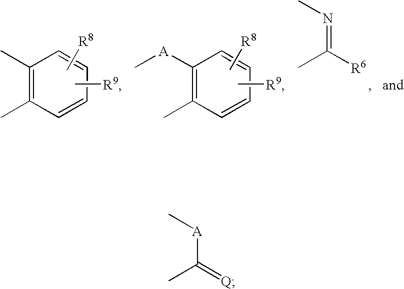
Q is selected from O and S;
A is selected from O, S, NR¹⁰, and CR¹⁰R¹⁰; and
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, C₁-C₄ heterohaloaliphatic, and a ring.

17. The method of claim 16, wherein Y is: 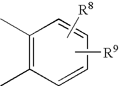
and at least one of R⁸ or R⁹ is C₁-C₄ haloaliphatic.

18. The method of claim 15, wherein R⁴ is hydrogen.

19. The method of claim 15, wherein R⁵ is OR¹⁰.

20. The method of claim 19, wherein R¹⁰ is hydrogen or a C₁-C₄ heteroalkyl.

21. The method of claim 15, wherein R⁷ is (CH₂)ₘR¹⁴, m is 0, and R¹⁴ is an optionally substituted aryl.

22. The method of claim 21, wherein R¹⁴ is an optionally substituted phenyl.

23. The method of claim 15, comprising reacting a compound having the structure: 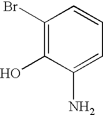
with a compound having the structure:

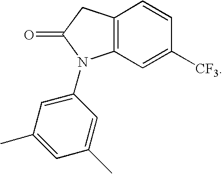

24. The method of claim 15, wherein said nitrite is sodium nitrite.

25. The method of claim 24, wherein the sodium nitrite is dissolved in an acidic solution.

26. The method of claim 25, wherein the acidic solution is an HCl solution.

27. A method of obtaining a compound having the structure: 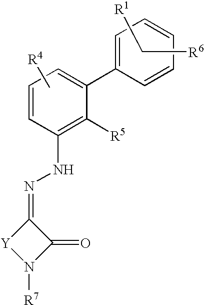
comprising reacting a compound having the structure: 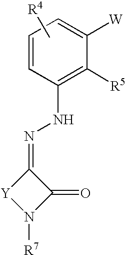
with a compound having the structure: 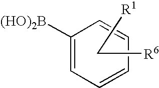
wherein:
R¹ is selected from CO₂R¹⁰, CONR¹⁰R¹¹, SO₃R¹⁰, and a carboxylic acid bioisostere;
R⁴ is selected from hydrogen, F, Cl, Br, C₁-C₄ aliphatic, C₁-C₄ haloaliphatic, C₁-C₄ heteroaliphatic, and a ring;
R⁵ is selected from hydrogen, OR¹⁰, SR¹⁰, NHR¹¹, and CO₂H;
R⁶ is selected from hydrogen, OR¹², NR¹²R¹³, F, Cl, Br, C₁-C₄ alkyl, C₁-C₄ haloalkyl, C₁-C₄ heteroalkyl, and a ring;
R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R¹⁰ is selected from hydrogen, a protecting group, an optionally substituted C₁-C₄ aliphatic, an optionally substituted C₁-C₄ haloaliphatic, an optionally substituted C₁-C₄ heteroaliphatic, and an optionally substituted ring;
R¹¹ is selected from hydrogen, SO₂R¹⁵, C₁-C₄ aliphatic, C₁-C₄ haloaliphatic, C₁-C₄ heteroaliphatic, and a ring;
R¹² and R¹³ are each independently selected from hydrogen, an optionally substituted C₁-C₄ aliphatic, an optionally substituted C₁-C₄ haloaliphatic, an optionally substituted C₁-C₄ heteroaliphatic, an optionally substituted ring, and (CH₂)ₘR¹⁴; or one of R¹² and R¹³ is an optionally substituted C₂-C₆ aliphatic or an optionally substituted ring and the other of R¹² and R¹³ is null; or R¹² and R¹³ are linked to form an optionally substituted C₃-C₈ ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
Y is a 1-4 atom spacer comprising one or more groups selected from an optionally substituted C₁-C₆ aliphatic, an optionally substituted C₁-C₆ heteroaliphatic, an optionally substituted phenyl, an optionally substituted heteroaryl, an optionally substituted C₃-C₅ heterocycle, and an optionally substituted alicyclic;
W is selected from a halogen, B(OH)₂, B(OR^{A})₂, Sn(R^{B})₃ where each R^{A} is selected from an optionally substituted C₁-C₆ aliphatic; or the two OR^{A} groups together form an optionally substituted ring; and R^{B} is selected from an optionally substituted C₁-C₆ aliphatic, or an optionally substituted phenyl, or an optionally substituted heteroaryl; and
m is 0, 1, or 2.

28. The method of claim 27, wherein:
Y is selected from: 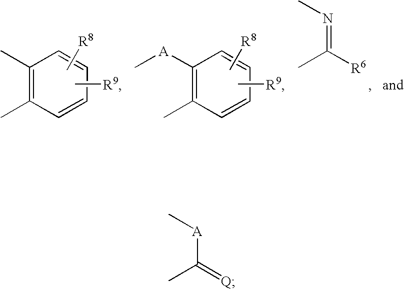
Q is selected from O and S;
A is selected from O, S, NR¹⁰, and CR¹⁰R¹⁰; and
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, C₁-C₄ heterohaloaliphatic, and a ring.

29. The method of claim 28, wherein R⁴ is hydrogen.

30. The method of claim 27, wherein Y is: 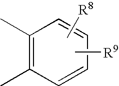
and at least one of R⁸ or R⁹ is C₁-C₄ haloaliphatic.

31. The method of claim 27, wherein R⁵ is OR¹⁰.

32. The method of claim 31, wherein R¹⁰ is hydrogen or a C₁-C₄ heteroalkyl.

33. The method of claim 27, wherein R⁷ is (CH₂)ₘR¹⁴, m is 0, and R¹⁴ is an optionally substituted aryl.

34. The method of claim 33, wherein R¹⁴ is an optionally substituted phenyl.

35. The method of claim 27, comprising reacting a compound having the structure: 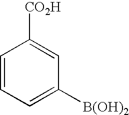
with a compound having the structure: 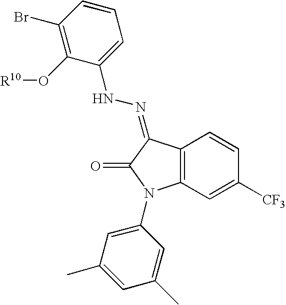
wherein R¹⁰ is hydrogen, a protecting group, an optionally substituted C₁-C₄ aliphatic, an optionally substituted C₁-C₄ haloaliphatic, or an optionally substituted C₁-C₄ heteroaliphatic.

36. The method of claim 27, wherein said reacting is conducted in the presence of a palladium catalyst.
